(19) United States
(12) Patent Application Publication
(10) Pub. No.: US 2026/0250658 A1
(43) Pub. Date: Aug. 27, 2026

(54) Lysis/Binding Solution, Cleaning Solution, Kit and Method for Nucleic Acid Extraction

(71) Applicants: BEIJING BOE TECHNOLOGY DEVELOPMENT CO., LTD., Beijing (CN); BOE TECHNOLOGY GROUP CO., LTD, Chaoyang District, Beijing (CN)

(72) Inventors: Zhihong Wu, Beijing (CN); Yaqi Zhang, Beijing (CN)

(21) Appl. No.: 18/992,163

(22) PCT Filed: Sep. 28, 2023
(86) PCT No.: PCT/CN2023/122798

§ 371 Date: Jan. 7, 2025

(51) Int. Cl. C12N 15/10; C12N 5/078; C12N 9/58

(52) U.S. Cl. CPC C12N 15/1013; C12N 5/0641; C12N 9/58; C12Y 304/21064

(57) ABSTRACT

A lysis/binding solution for nucleic acid extraction is provided. The lysis/binding solution for nucleic acid extraction includes a protein denaturant, a non-alcohol dehydrating agent, a first surfactant, a buffer solution, ethylenediaminetetraacetic acid, and a chaotropic salt. A molarity of the protein denaturant ranges from 3.5 mol/L to 5.5 mol/L, a weight by volume percent of the non-alcohol dehydrating agent ranges from 0.1% to 1%, a weight by volume percent of the first surfactant ranges from 0.05% to 0.5%, a molarity of the buffer solution ranges from 5 mmol/L to 150 mmol/L, a molarity of the ethylenediaminetetraacetic acid ranges from 5 mmol/L to 20 mmol/L, and a weight by volume percent of the chaotropic salt ranges from 1% to 8%.

### CROSS-REFERENCE TO RELATED APPLICATION

This application is the United States national phase of International Patent Application No. PCT/CN2023/122798 filed Sep. 28, 2023, the disclosure of which is hereby incorporated by reference in its entirety.

### BACKGROUND OF THE INVENTION

### Field of the Invention

The present disclosure relates to the field of molecular diagnosis technology, and in particular, to a lysis/binding solution, a cleaning solution, a kit and a method for nucleic acid extraction.

### Description of Related Art

With nucleic acids as the detection object, molecular diagnosis is mainly applied in the diagnosis of various clinical departments, such as tumor, infection, and genetics. The basis of all work is nucleic acid extraction. Under specific conditions, the surface of magnetic beads as an adsorption carrier is encapsulated with groups such as silicon, amino or carboxyl groups, or carriers such as solid-phase silica membranes. Relying on electrostatic, hydrophobic and hydrogen bonding interactions, the specific binding of nucleic acids to magnetic beads can be achieved. Non-specific impurities, salts, etc., can be removed through several washings, and then the nucleic acids can be eluted from the adsorption carrier for purification.

### SUMMARY OF THE INVENTION

In an aspect, a lysis/binding solution for nucleic acid extraction is provided. The lysis/binding solution for nucleic acid extraction includes: a protein denaturant, a non-alcohol dehydrating agent, a first surfactant, a buffer solution, ethylenediaminetetraacetic acid, and a chaotropic salt. A molarity of the protein denaturant ranges from 3.5 mol/L to 5.5 mol/L, a weight by volume percent of the non-alcohol dehydrating agent ranges from 0.1% to 1%, a weight by volume percent of the first surfactant ranges from 0.05% to 0.5%, a molarity of the buffer solution ranges from 5 mmol/L to 150 mmol/L, a molarity of the ethylenediaminetetraacetic acid ranges from 5 mmol/L to 20 mmol/L, and a weight by volume percent of the chaotropic salt ranges from 1% to 8%.

In some embodiments, anions in the chaotropic salt include at least one type of: PO₄³⁻, SO₄²⁻, H₂PO₄⁻, HCOO⁻, Cl⁻, NO₃⁻, CF₃COO⁻, BF₄⁻, ClO₄⁻, and PF₆⁻.

In some embodiments, cations in the chaotropic salt include at least one type of: Ca²⁺, Mg²⁺, Li⁺, Na⁺, K⁺, NH₄⁺, an imidazolium cation, and an N-ethylpyridinium cation.

In some embodiments, the chaotropic salt includes at least one of: ammonium phosphate, ammonium sulfate, ammonium dihydrogen phosphate, ammonium chloride, calcium hexafluorophosphate, potassium hexafluorophosphate, sodium hexafluorophosphate, ammonium hexafluorophosphate, 1-allyl-3-methylimidazolium hexafluorophosphate, N-ethylpyridinium hexafluorophosphate, and N-ethylpyridinium chloride.

In some embodiments, the non-alcohol dehydrating agent includes at least one of: polyoxyethylene lauryl ether, polyvinyl carbazole, polyvinyl alcohol, polyacrylamide, polyacrylic acid, polyoxyethylene sorbitan monolaurate, and sodium dodecyl sulfate.

In some embodiments, the protein denaturant includes at least one of: guanidine isothiocyanate and guanidine hydrochloride.

In some embodiments, the buffer solution includes at least one of: a Tris-EDTA (TE) buffer solution and tris (hydroxymethyl)aminomethane, the TE buffer solution comprising tris (hydroxymethyl)aminomethane hydrochloride and ethylenediaminetetraacetic acid.

In some embodiments, the first surfactant includes polyoxyethylene sorbitan monolaurate or polyethylene glycol-p-isooctylphenyl ether.

In some embodiments, pH of the lysis/binding solution takes a value ranging from 4.4 to 7.4.

In some embodiments, the protein denaturant is guanidine isothiocyanate, the non-alcohol dehydrating agent is sodium dodecyl sulfate, the first surfactant is polyoxyethylene sorbitan monolaurate, the buffer solution is a TE buffer solution, and the chaotropic salt is N-ethylpyridinium hexafluorophosphate. A molarity of the guanidine isothiocyanate is 5 mol/L, a weight by volume percent of the sodium dodecyl sulfate is 0.5%, a weight by volume percent of the polyoxyethylene sorbitan monolaurate is 0.1%, a molarity of the TE buffer solution is 100 mmol/L, the molarity of the ethylenediaminetetraacetic acid is 10 mmol/L, and a weight by volume percent of the N-ethylpyridinium hexafluorophosphate is 2%; and pH of the lysis/binding solution takes a value of 7.4.

In some embodiments, pH of the buffer solution takes a value ranging from 7.0 to 7.4.

In some embodiments, pH of the buffer solution takes a value of 7.2.

In another aspect, a cleaning solution for nucleic acid extraction is provided. The cleaning solution for nucleic acid extraction includes: sodium chloride and a second surfactant, where a molarity of the sodium chloride ranges from 0.05 mol/L to 0.5 mol/L, and a weight by volume percent of the second surfactant ranges from 0.5% to 5%; and pH of the cleaning solution takes a value ranging from 6.9 to 7.1.

In some embodiments, the second surfactant includes at least one of: polyethylene glycol p-isooctylphenyl ether, polyoxyethylene sorbitan monolaurate, and ethylphenyl polyethylene glycol.

In yet another aspect, a kit for nucleic acid extraction is provided. The kit for nucleic acid extraction includes: the lysis/binding solution for nucleic acid extraction as described in any of the above embodiments; and the kit for nucleic acid extraction further includes: a second cleaning solution, including the cleaning solution as described in any of the above embodiments.

In some embodiments, the kit for nucleic acid extraction further includes: a red blood cell lysis solution. The red blood cell lysis solution includes: sodium chloride, polyoxyethylene sorbitan monolaurate, glucose, and tris (hydroxymethyl)aminomethane, where a molarity of the sodium chloride ranges from 1 mol/L to 10 mol/L, a weight by volume percent of the polyoxyethylene sorbitan monolaurate ranges from 3% to 6%, a molarity of the glucose ranges from 200 mmol/L to 500 mmol/L, and a molarity of the tris (hydroxymethyl)aminomethane ranges from 1 mmol/L to 50 mmol/L; and pH of the red blood cell lysis solution takes a value ranging from 8.0 to 8.4.

In some embodiments, the kit for nucleic acid extraction further includes: a proteinase K solution with a concentration ranging from 10 mg/mL to 30 mg/mL.

In some embodiments, the kit for nucleic acid extraction further includes: a magnetic bead suspension with a concentration ranging from 10 mg/mL to 40 mg/mL.

In some embodiments, the kit for nucleic acid extraction further includes: a first cleaning solution. The first cleaning solution includes: guanidine isothiocyanate, tris (hydroxymethyl)aminomethane hydrochloride, polyoxyethylene sorbitan monolaurate, sodium chloride, and isopropanol, where a molarity of the guanidine isothiocyanate ranges from 0.5 mol/L to 3 mol/L, a molarity of the tris (hydroxymethyl)aminomethane hydrochloride ranges from 0.5 mmol/L to 10 mmol/L, a weight by volume percent of the polyoxyethylene sorbitan monolaurate ranges from 0.01% to 0.1%, a molarity of the sodium chloride ranges from 0.5 mol/L to 2 mol/L, and a percent by volume of the isopropanol ranges from 20% to 50%; and pH of the first cleaning solution takes a value ranging from 6.5 to 8.0.

In some embodiments, the kit for nucleic acid extraction further includes: an eluent solution, where the eluent solution includes: a TE buffer solution.

In still another aspect, a method for nucleic acid extraction is provided. The method for nucleic acid extraction included: adding a lysis/binding solution and a proteinase K solution to a sample to be performed with nucleic acid extraction and mixing the lysis/binding solution, the proteinase K solution and the sample to form a mixed solution, at a temperature condition of 55° C. to 80° C.; adding a magnetic bead suspension to the mixed solution, mixing the magnetic bead suspension and the mixed solution, placing the magnetic bead suspension and the mixed solution on a magnetic rack for standing, and removing a supernatant to obtain a second precipitate; adding a first cleaning solution to the second precipitate, mixing the first cleaning solution and the second precipitate, placing the first cleaning solution and the second precipitate on a magnetic rack for standing, and removing a supernatant, to obtain a third precipitate; adding a second cleaning solution to the third precipitate, mixing the second cleaning solution and the third precipitate, placing the second cleaning solution and the third precipitate on a magnetic rack for standing, and removing a supernatant to obtain a fourth precipitate; and adding an eluent solution to the fourth precipitate and mixing the eluent solution and the fourth precipitate, at a temperature condition of 40° C. to 60° C., placing the eluent solution and the fourth precipitate on a magnetic rack for standing, and removing a supernatant to obtain extracted nucleic acids.

In some embodiments, a volume of the lysis/binding solution is 2 to 4 times a volume of a blood sample, a volume of the proteinase K solution is 0.05 to 0.2 times the volume of the blood sample, a volume of the magnetic bead suspension is 0.075 to 0.2 times the volume of the blood sample, a volume of the first cleaning solution is 2.4 to 4.5 times the volume of the blood sample, and a volume of the second cleaning solution is 2.4 to 4.5 times the volume of the blood sample; and the sample to be performed with nucleic acid extraction is obtained through processing the blood sample.

In some embodiments, the method for nucleic acid extraction further includes: before adding the lysis/binding solution and the proteinase K solution to the sample and mixing the lysis/binding solution, the proteinase K solution and the sample, putting a blood sample in a container and adding a red blood cell lysis solution in the container, mixing the blood sample and the red blood cell lysis solution and performing centrifugation thereon, and removing a supernatant to obtain a first precipitate, the sample to be performed with nucleic acid extraction being the first precipitate. A rotational speed of centrifugation ranges from 10000 rpm to 12000 rpm, and a time of centrifugation ranges from 1 minute to 3 minutes; and adding the red blood cell lysis solution, mixing the blood sample and the red blood cell lysis solution and performing centrifugation thereon, and removing the supernatant are repeated at least once; and a volume of the red blood cell lysis solution is 3 to 5 times a volume of the blood sample.

### BRIEF DESCRIPTION OF THE DRAWINGS

In order to describe technical solutions in the present disclosure more clearly, the accompanying drawings to be used in some embodiments of the present disclosure will be introduced briefly. It is obvious that the accompanying drawings to be described below are merely drawings of some embodiments of the present disclosure, and a person of ordinary skill in the art can obtain other drawings according to those drawings. In addition, the accompanying drawings in the following description may be regarded as schematic diagrams, but are not limitations on actual sizes of products, actual processes of methods and actual timings of signals involved in the embodiments of the present disclosure.

FIG. 1 is a flowchart of a nucleic acid extraction provided in accordance with some embodiments of the present disclosure;

FIG. 2 is a histogram showing the efficiency of a nucleic acid extraction provided in accordance with some embodiments of the present disclosure;

FIG. 3 is a scattergram showing the purity of a nucleic acid extraction provided in accordance with some embodiments of the present disclosure;

FIG. 4 is a gel electropherogram for a nucleic acid extraction provided in accordance with some embodiments of the present disclosure;

FIG. 5 is a histogram showing the efficiency of another nucleic acid extraction provided in accordance with some embodiments of the present disclosure;

FIG. 6 is a scattergram showing the purity of another nucleic acid extraction provided in accordance with some embodiments of the present disclosure;

FIG. 7 is a gel electropherogram for another nucleic acid extraction provided in accordance with some embodiments of the present disclosure;

FIG. 8 is a line graph of the yield of a nucleic acid extraction provided in accordance with some embodiments of the present disclosure;

FIG. 9 is a line graph of the yield of another nucleic acid extraction provided in accordance with some embodiments of the present disclosure;

FIG. 10 is a line graph of the yield of yet another nucleic acid extraction provided in accordance with some embodiments of the present disclosure; and

FIG. 11 is a gel electropherogram for yet another nucleic acid extraction provided in accordance with some embodiments of the present disclosure.

### DESCRIPTION OF THE INVENTION

The technical solutions in some embodiments of the present disclosure will be described clearly and completely with reference to the accompanying drawings. It is obvious that the described embodiments are merely some but not all embodiments of the present disclosure. All other embodiments obtained by a person of ordinary skill in the art based on embodiments of the present disclosure shall be included in the protection scope of the present disclosure.

Unless the context requires otherwise, throughout the specification and the claims, the term “comprise” and other forms thereof such as the third-person singular form “comprises” and the present participle form “comprising” are construed as an open and inclusive meaning, i.e., “including, but not limited to.” In the description of the specification, the terms such as “one embodiment,” “some embodiments,” “exemplary embodiments,” “example,” “specific example,” or “some examples” are intended to indicate that specific features, structures, materials, or characteristics related to the embodiment(s) or example(s) are included in at least one embodiment or example of the present disclosure. Schematic representations of the above terms do not necessarily refer to the same embodiment(s) or example(s). In addition, the specific features, structures, materials or characteristics may be included in any one or more embodiments or examples in any suitable manner.

Hereinafter, the terms such as “first” and “second” are used for descriptive purposes only, but are not to be construed as indicating or implying the relative importance or implicitly indicating the number of indicated technical features. Thus, the features defined by “first” and “second” may explicitly or implicitly include one or more of the features. In the description of the embodiments of the present disclosure, the term “a plurality of” means two or more unless otherwise specified.

The phrase “at least one of A, B and C” has the same meaning as the phrase “at least one of A, B or C,” and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

The term such as “about,” “substantially” or “approximately” as used herein includes a stated value and an average value within an acceptable range of deviation of a particular value determined by a person of ordinary skill in the art, considering measurement in question and errors associated with measurement of a particular quantity (i.e., limitations of a measurement system).

The term such as “parallel,” “perpendicular” or “equal” as used herein includes a stated case and a case similar to the stated case within an acceptable range of deviation determined by a person of ordinary skill in the art, considering measurement in question and errors associated with measurement of a particular quantity (i.e., limitations of a measurement system). For example, the term “parallel” includes absolute parallelism and approximate parallelism, and an acceptable range of deviation of the approximate parallelism may be, for example, a deviation within 5°; the term “perpendicular” includes absolute perpendicularity and approximate perpendicularity, and an acceptable range of deviation of the approximate perpendicularity may also be, for example, a deviation within 5°; and the term “equal” includes absolute equality and approximate equality, and an acceptable range of deviation of the approximate equality may be, for example, that a difference between two equals is less than or equal to 5% of either of the two equals.

As used herein, the term “kit” is a box used to contain chemical reagents for detecting chemical components, drug residues, virus types and the like. Of course, those skilled in the art can understand that the box may also be any of other containers for containing chemical reagents, such as a tube.

As used herein, the term “DNA” is an abbreviation for deoxyribonucleic acid. DNA, as a carrier of genetic information in biological cells, is mainly used to guide synthesis of RNA and proteins in a body. DNA is a macromolecular polymer composed of deoxynucleotides, and the deoxynucleotide is composed of a phosphate group, a deoxyribose, and a base, in which there are four types of bases, namely A (Adenine), G (Guanine), C (Cytosine), and T (Thymine).

As used herein, the term “RNA” is an abbreviation for ribonucleic acid. The RNA, as a carrier of genetic information that exists in biological cells and some viruses and viroids, is mainly used to guide synthesis of proteins in the body. RNA is a macromolecular polymer composed of ribonucleotides, and the ribonucleotide is composed of a phosphate group, a ribose, and a base, in which there are four types of bases, namely A (Adenine), G (Guanine), C (Cytosine), and U (Uracil). As used herein, the term “nucleic acid” includes DNA and/or RNA.

As used herein, the term “PCR” is an abbreviation for polymerase chain reaction. PCR is a molecular biology technology used to amplify and replicate specific DNA fragments, which can be viewed as a special DNA replication for organisms in vitro, and has the greatest feature of being able to significantly increase a minute amount of DNA.

As used herein, the term “NGS” is an abbreviation for high-throughput sequencing, also known as “next-generation” sequencing technology, characterized by the ability to sequence hundreds of thousands to millions of DNA molecules at one time in parallel and a relatively short read length in a single sequencing.

Nucleic acid extraction technology is the foundation of modern molecular diagnostic technology, and its extraction efficiency and purity have a significant impact on the results of downstream experiments, such as PCR (Polymerase Chain Reaction) and NGS (High-Throughput Sequencing). With the development and popularization of molecular diagnostic technology, especially since the outbreak of the COVID-19 pandemic, the number of samples for nucleic acid testing has increased significantly. The traditional manual nucleic acid extraction method can no longer meet the actual testing needs. Therefore, the manual nucleic acid extraction method is gradually being replaced by the automatic nucleic acid extraction method.

Currently, the main method for automatic nucleic acid extraction includes a magnetic rod method, in which the basic steps of extraction include that: 1), lysis of biological samples (such as viruses) and release of nucleic acids in a sample, e.g., the sample being a blood sample; 2), nucleic acids and magnetic beads are bound under certain buffer solution conditions; 3), nucleic acids are cleaned to remove impurities; and 4), nucleic acids are released from an adsorption carrier into the solution, e.g., the adsorption carrier being magnetic beads.

In step 1), cell membranes of the biological samples break down and digest proteins, so proteinase K is used in this process. A binding solution in step 2) contains isopropanol, where isopropanol is a commonly used nucleic acid precipitation reagent, and isopropanol itself will not damage nucleic acids. However, alcohol solvents will have a certain inhibitory effect on enzymes, that is, isopropanol will inhibit the activity of proteinase K. Therefore, in the conventional nucleic acid extraction process, components of the lysis solution are separated from components of the binding solution, and the binding solution is added for magnetic bead capture after the lysis step is fully completed.

That is, the lysis process of the biological samples in step 1) and the binding process of nucleic acids and magnetic beads in step 2) are performed separately. Moreover, the magnetic rod extractor is usually operated only by adsorbing, moving, and releasing the magnetic beads. This means that in actual use, it is necessary to wait for the biological samples to be lysed and then manually add the binding solution to a slot containing the lysis solution, making it impossible to truly realize a fully automatic process.

In addition, as the operator repeatedly takes and places the sample from the slot, there exists the possibility that the sample receives contamination from the operator; moreover, as the interior of the extraction chamber is closed, the internal gas does not flow, and the external air passes directly into the extractor is prone to breed bacteria, which pollute the interior of the extractor, thereby reducing the efficiency and quality of nucleic acid extraction.

Furthermore, the automatic extractor uses a kit slot pre-filled with reagents, and components of a cleaning solution therein usually contain ethanol, which is volatile. The volatilization of ethanol will cause the proportion of the components to deviate, which is not conducive to the long-term preservation of the kit slot.

In light of this, embodiments of the present disclosure provide a lysis/binding solution for nucleic acid extraction. The lysis/binding solution for nucleic acid extraction includes: a protein denaturant, a non-alcohol dehydrating agent, a first surfactant, a buffer solution, ethylenediaminetetraacetic acid, and a chaotropic salt, where a molarity of the protein denaturant ranges from 3.5 mol/L to 5.5 mol/L, a weight by volume percent of the non-alcohol dehydrating agent ranges from 0.1% to 1%, a weight by volume percent of the first surfactant ranges from 0.05% to 0.5%, a molarity of the buffer solution ranges from 5 mmol/L to 150 mmol/L, a molarity of ethylenediaminetetraacetic acid ranges from 5 mmol/L to 20 mmol/L, and a weight by volume percent of the chaotropic salt ranges from 1% to 8%.

The lysis/binding solution is used to release nucleic acids from biological samples (such as viruses) in blood samples and to destroy the protein structure of the biological samples (such as viruses), thereby promoting the separation of proteins and nucleic acids, enabling nucleic acid molecules to be dehydrated, and promoting the nucleic acids to be effectively bound to an adsorption carrier (such as magnetic beads).

For example, the protein denaturant includes at least one of guanidine isothiocyanate and guanidine hydrochloride.

It will be noted that the function of the protein denaturant is to destroy protein molecules of the viruses in the sample. The main function of the non-alcohol dehydrating agent is to prevent DNA from being too soluble in water, which will result in the DNA being unable to be completely adsorbed by the magnetic beads and thus being lost. The dehydration principle of the non-alcohol dehydrating agent is to reduce the solubility of DNA in water through polarity compatibility competition between the non-alcohol dehydrating agent and water molecules.

The chaotropic salt is a salt capable of disrupting hydrogen bonds between water molecules, increasing the disorder of water molecules. The main function of chaotropic salt is to destroy the cell membrane and nuclear membrane structure in the sample, mainly by destroying hydrogen bonds and disulfide bonds inside the protein, thereby destroying its secondary structure and causing the interaction between proteins, and between proteins and DNA to be greatly weakened, and thereby destroying the membrane structure of biological samples (such as viruses) and separating DNA, destroying the binding of nucleoproteins and DNA, and enabling DNA to be adsorbed to the magnetic beads.

If the non-alcohol dehydrating agent has too high content, it will affect the solubility of the solution, resulting in the non-alcohol dehydrating agent not being completely dissolved in water; and the non-alcohol dehydrating agent with too high content will be encapsulated on the surface of the magnetic beads, affecting the adsorption of DNA by the magnetic beads. The chaotropic salt with an appropriate concentration can break hydrogen bonds between water molecules and enhance the hydrophobicity of DNA. Moreover, combined with the non-alcohol dehydrating agent with an appropriate concentration, the chaotropic salt with an appropriate concentration can make nucleic acids precipitate out from an aqueous solution to bind with the magnetic beads. In this way, the amount of the non-alcohol dehydrating agent used can be reduced, and the chaotropic salt can further cooperate with molecules of the protein denaturant (e.g., guanidine isothiocyanate) to destroy the protein molecules of the biological samples (such as viruses) to further reduce protein residues and improve the purity of extracted DNA. Moreover, the dehydrating agent has a certain solubility for polysaccharides and lipid groups inside cells, which can reduce the interference of impurities and promote the destruction of the secondary structure of the protein by the chaotropic salt. The non-alcohol dehydrating agent further encapsulates the protein molecules, and then the protein molecules are carried away by solution transfer, thereby reducing protein impurities in the solution.

The main function of the first surfactant is to increase the dispersion of the magnetic beads in an aqueous solution and facilitate the adsorption of DNA. The buffer solution provides a liquid system for dissolving nucleic acids.

Ethylenediaminetetraacetic acid is abbreviated as EDTA, with a molecular formula of C₁₀H₁₆N₂O₈. Ethylenediaminetetraacetic acid is a good complexing agent with six coordinating atoms, forming a complex called a chelate. EDTA is often used in coordination titration, generally to determine the content of a metal ion. In biological applications, ethylenediaminetetraacetic acid is used to eliminate the interference of most transition metal ions (such as iron (Group VIIIb), nickel (Group VIIIb), and manganese (Group IIA)). Ethylenediaminetetraacetic acid here acts as a chelator for enzyme-catalyzed ions (mainly magnesium ions).

For example, the molarity of the protein denaturant is 3.5 mol/L, 4.0 mol/L, 4.5 mol/L, 5.0 mol/L, or 5.5 mol/L, which is not limited here.

For example, the weight by volume percent of the non-alcohol dehydrating agent is 0.1%, 0.2%, 0.3%, 0.4%, 0.5%, 0.6%, 0.7%, 0.8%, 0.9%, or 1%, which is not limited here.

It will be noted that the “weight by volume percent” is a ratio of the weight of a substance (in grams) to the volume of a liquid (in milliliters). For example, the weight by volume percent of the non-alcohol dehydrating agent is 1%, which means that the weight of the non-alcohol dehydrating agent is 1 gram in 100 mL of the lysis/binding solution.

For example, the weight by volume percent of the first surfactant is 0.05%, 0.1%, 0.15%, 0.2%, 0.25%, 0.3%, 0.35%, 0.4%, 0.45%, or 5%, which is not limited here.

For example, the molarity of the buffer solution is 5 mmol/L, 10 mmol/L, 15 mmol/L, 20 mmol/L, 40 mmol/L, 55 mmol/L, 65 mmol/L, 70 mmol/L, 75 mmol/L, 85 mmol/L, 90 mmol/L, 100 mmol/L, 120 mmol/L, or 150 mmol/L, which is not limited here.

For example, the molarity of ethylenediaminetetraacetic acid is 5 mmol/L, 7 mmol/L, 9 mmol/L, 10 mmol/L, 13 mmol/L, 16 mmol/L, or 20 mmol/L, which is not limited here.

For example, the weight by volume percent of the chaotropic salt is 1%, 2%, 3%, 4%, 5%, 6%, 7%, or 8%, which is not limited here.

For example, Pondus Hydrogenii (pH) of the lysis/binding solution takes a value ranging from 4.4 to 7.4. The pH of the lysis/binding solution takes a value of 4.4, 4.6, 5.2, 5.8, 6.0, 6.3, 6.5, 6.8, 7.2, or 7.4, which is not limited here. Adjusting the value of the pH of the lysis/binding solution to weak acidity can enhance the hydrogen bonding ability between the surface of the magnetic beads and the nucleic acids, thereby enabling the nucleic acids to quickly bind to the surface of the magnetic beads without affecting the digestion ability of proteinase K, thereby implementing the lysis process and the binding process in a single step.

Therefore, the embodiments of the present disclosure use a non-isopropanol system reagent to unify the components of the lysis solution and the binding solution, thereby reducing the time of nucleic acid extraction, enabling the sample to be bound to the magnetic beads while being lysed, and better matching the automatic nucleic acid extractor to realize fully automatic extraction.

In some examples, anions in the chaotropic salt include at least one type of: PO₄³, SO₄²⁻, H₂PO₄⁻, HCOO⁻, Cl⁻, NO₃⁻, CF₃COO⁻, BF₄⁻, ClO₄⁻, and PF₆⁻. In some examples, cations in the chaotropic salt include at least one type of: Ca²⁺, Mg²⁺, Li⁺, Na⁺, K⁺, NH₄⁺, an imidazolium cation, and an N-ethylpyridinium cation.

It will be noted that the imidazolium cation has a structure shown in the following structural formula I, where R₁ and R₂ each include an alkyl group:

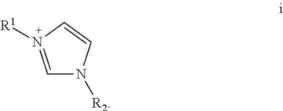

For example, two types of imidazolium cations are exemplified below:

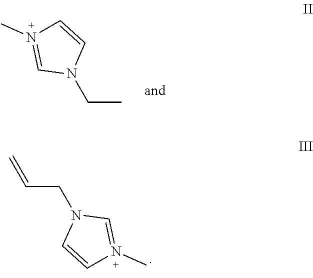

Here, in the structural formula II, R₁ is methyl and R₂ is ethyl; and in the structural formula III, R₁ is methyl and R₂ is propenyl.

The structural formula of N-ethylpyridinium cation is shown below:

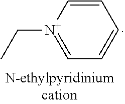

For example, the chaotropic salt includes ammonium phosphate, ammonium sulfate, ammonium dihydrogen phosphate, ammonium chloride, calcium hexafluorophosphate, potassium hexafluorophosphate, sodium hexafluorophosphate, ammonium hexafluorophosphate, 1-allyl-3-methylimidazolium hexafluorophosphate, N-ethylpyridinium hexafluorophosphate, or N-ethylpyridinium chloride, which is not limited here.

Ammonium phosphate has the molecular formula of (NH₄)₃PO₄, existing in a form of PO₄³⁻ and NH₄⁺ in an aqueous solution.

Ammonium sulfate has the molecular formula of (NH₄)₂SO₄, existing in a form of SO₄²⁻ and NH₄⁺ in an aqueous solution.

Ammonium dihydrogen phosphate has the molecular formula of NH₄H₂PO₄, existing in a form of H₂PO₄⁻ and NH₄⁺ in an aqueous solution.

Ammonium chloride has the molecular formula of NH₄Cl, existing in a form of Cl⁻ and NH₄⁺ in an aqueous solution.

Calcium hexafluorophosphate has the molecular formula of CaF₁₂P₂, existing in a form of PF₆ and Ca²⁺ in an aqueous solution.

Potassium hexafluorophosphate has the molecular formula of KPF₆, existing in a form of PF₆⁻ and K⁺ in an aqueous solution.

Sodium hexafluorophosphate has the molecular formula of NaPF₆, which is colorless crystalline powder and an inorganic compound, existing in a form of PF₆⁻ and Na⁺ in an aqueous solution.

Ammonium hexafluorophosphate has the molecular formula of NH₄PF₆, which is white crystalline powder and an inorganic compound, existing in a form of PF₆ and NH₄⁺ in an aqueous solution.

1-allyl-3-methylimidazolium hexafluorophosphate has the molecular formula of C₉H₁₁PF₆N₂, existing in a form of PF₆ and an imidazolium cation in an aqueous solution, where the structure of the imidazolium cation is shown in the structural formula III.

N-ethylpyridinium hexafluorophosphate has the molecular formula of C₇H₁₀F₆NP, existing in a form of PF₆⁻ and an N-ethylpyridinium cation in an aqueous solution.

N-ethylpyridinium chloride has the molecular formula of C₇H₁₀ClN, existing in a form of Cl⁻ and an N-ethylpyridinium cation in an aqueous solution.

In some examples, the non-alcohol dehydrating agent includes at least one of: polyoxyethylene lauryl ether, polyvinyl carbazole (PVK), polyvinyl alcohol, polyacrylamide, polyacrylic acid, polyoxyethylene sorbitan monolaurate, and sodium dodecyl sulfate.

Since the presence of the non-alcohol dehydrating agent needs not to affect the solubility of proteins, a polar organic substance is selected as the dehydrating agent, enabling the non-alcohol dehydrating agent and the chaotropic salt to maintain a certain degree of mutual solubility in an aqueous solution.

Polyoxyethylene lauryl ether, having the molecular formula of C₃₈H₇₆O₁₁, is a nonionic surfactant and has polar groups in a form of polyoxyethylene separated by a polyethylene chain. The molecular structure of polyoxyethylene lauryl ether consists of a single chain of polyethylene glycol (PEG). Polyoxyethylene lauryl ether is slightly soluble in water and is hydrophobic, and polyoxyethylene lauryl ether is a brown viscous liquid, easily soluble in water, and has emulsification, wetting and dispersing abilities.

Polyvinyl carbazole has the molecular formula of C₄₂H₃₃N₃X₂. Poly(N-vinyl carbazole), i.e., Polyvinyl carbazole, is a colorless-transparent or brown-transparent amorphous thermoplastic resin. The carbazole group imparts high thermal stability, water resistance and chemical stability to the resin. Polyvinyl carbazole is insoluble in aliphatic hydrocarbons, mineral oil, transformer oil, castor oil, carbon tetrachloride, ethanol, ether, dilute acid, hydrofluoric acid, etc., and is easily soluble in concentrated sulfuric acid, concentrated nitric acid, tetrahydrofuran, and chlorinated hydrocarbons.

Polyvinyl alcohol has the molecular formula of (C₂H₄O)ₙ, where n is a positive integer. The average molecular weight of polyvinyl alcohol ranges from 16000 to 20000. Polyvinyl alcohol is a white flake, flocculent or powdery solid and is odorless. Polyvinyl alcohol is soluble in water, insoluble in gasoline, kerosene, vegetable oil, benzene, toluene, ethylene dichloride, carbon tetrachloride, acetone, ethyl acetate, methanol, ethylene glycol, etc., and slightly soluble in dimethyl sulfoxide.

Polyacrylamide has the molecular formula of (C₃H₅NO)ₙ, where n is a positive integer. Polyacrylamide is a linear organic macromolecular polymer and also a macromolecular water treatment flocculant product, which can specifically adsorb suspended particles in water, act as a link and bridge between particles, make fine particles form relatively large flocs, so as to accelerate the rate of precipitation.

Polyacrylic acid has the molecular formula of (C₃H₄O₂)ₙ, where n is a positive integer, which is used to prepare finishing agents for leather and certain high-end products, to prepare acrylic resin paints, etc., and is a chemical intermediate.

Polyoxyethylene sorbitan monolaurate has the molecular formula of C₂₆H₅₀O₁₀, also known as Tween 20, which is a surfactant, i.e., a type of macromolecule with both hydrophilic and lipophilic parts. Therefore, polyoxyethylene sorbitan monolaurate can promote plants to absorb large molecules that are insoluble in water, and can also help molecules of water pass through some biological membranes with high lipid content.

Sodium dodecyl sulfate, referred to as SDS, has the molecular formula of C₁₂H₂₅SO₄Na, which is a white or light yellow slightly sticky substance and is commonly used in detergents and the textile industry. Sodium dodecyl sulfate is an anionic surfactant, easily soluble in water, has good compatibility with anions and non-ions, and has good emulsification, foaming, penetration, cleansing and dispersing properties.

In some examples, the buffer solution includes at least one of: a Tris-EDTA (TE) buffer solution and tris (hydroxymethyl)aminomethane. The TE buffer solution includes 10 mmol/L tris (hydroxymethyl)aminomethane hydrochloride and 0.1 mmol/L ethylenediaminetetraacetic acid.

For example, pH of the buffer solution ranges from 7.0 to 7.4. For example, the pH of the buffer solution takes a value of 7.0, 7.1, 7.2, 7.3, or 7.4, which is not limited here.

Tris(hydroxymethyl)aminomethane is abbreviated as Tris, having the molecular formula of C₄H₁₁NO₃, which is a biological buffer used to prepare buffer solution for gel electrophoresis.

Tris (hydroxymethyl)aminomethane hydrochloride is abbreviated as Tris-HCl, having the molecular formula of C₄H₁₁NO₃·HCl.

For the description of ethylenediaminetetraacetic acid, please refer to the above content and will not be repeated here.

In some examples, the first surfactant includes polyoxyethylene sorbitan monolaurate or polyethylene glycol-p-isooctylphenyl ether.

For the description of polyoxyethylene sorbitan monolaurate, please refer to the above content and will not be repeated here.

Polyethylene glycol-p-isooctylphenyl ether is abbreviated as Triton X-100, having the molecular formula of C₁₆H₂₆O₂.

A kit for nucleic acid extraction is introduced below.

The embodiments of the present disclosure provide a kit for nucleic acid extraction. The kit for nucleic acid extraction includes: a red blood cell lysis solution, a lysis/binding solution for nucleic acid extraction, a proteinase K solution, a magnetic bead suspension, a first cleaning solution, a second cleaning solution, and an elution solution.

The red blood cell lysis solution is used to destroy red blood cells in the blood. In some examples, the red blood cell lysis solution includes: sodium chloride, polyoxyethylene sorbitan monolaurate, glucose, and tris (hydroxymethyl)aminomethane. Here, a molarity of sodium chloride ranges from 1 mol/L to 10 mol/L, a weight by volume percent of polyoxyethylene sorbitan monolaurate ranges from 3% to 6%, a molarity of glucose ranges from 200 mmol/L to 500 mmol/L, and a molarity of tris (hydroxymethyl)aminomethane ranges from 1 mmol/L to 50 mmol/L. pH of the red blood cell lysis solution takes a value ranging from 8.0 to 8.4.

Sodium chloride is used to ensure the osmotic pressure between white blood cells and an aqueous solution, preventing white blood cells from being osmotically ruptured by the aqueous solution during the lysis process of red blood cells.

For example, the molarity of sodium chloride is 1 mol/L, 2 mol/L, 3 mol/L, 4 mol/L, 5 mol/L, 6 mol/L, 7 mol/L, 8 mol/L, 9 mol/L, or 10 mol/L, which is not limited here.

Polyoxyethylene sorbitan monolaurate is used to allow membrane fragments, lipid small molecules and other organic substances from lysed red blood cells to be eluted from the solution.

For example, the weight by volume percent of polyoxyethylene sorbitan monolaurate is 3%, 4%, 5%, or 6%, which is not limited here.

Glucose is used to destroy the cell membranes of red blood cells.

For example, the molarity of glucose is 200 mmol/L, 250 mmol/L, 300 mmol/L, 350 mmol/L, 400 mmol/L, 450 mmol/L, or 500 mmol/L, which is not limited here.

The molarity of tris (hydroxymethyl)aminomethane is 1 mmol/L, 10 mmol/L, 20 mmol/L, 25 mmol/L, 30 mmol/L, 40 mmol/L, or 50 mmol/L, which is not limited here.

For blood samples, the red blood cells can be first swelled and lysed using the concentrated salt of the red blood cell lysis solution, and then the white blood cells can be lysed after separation, which can prevent nucleic acids from being contaminated in subsequent steps due to the introduction of a large amount of sugar and proteins on the surface of the red blood cells.

For the description of the lysis/binding solution for nucleic acid extraction, please refer to the above content and will not be repeated here.

Proteinase K is a powerful protein-solubilizing enzyme isolated from Candida albicans with high specific activity and is a key reagent for DNA extraction. The proteinase K solution is used for breaking the walls of tissue cells. For example, a concentration of the proteinase K solution ranges from 10 mg/mL to 30 mg/mL. For example, the concentration of the proteinase K solution is 10 mg/mL, 13 mg/mL, 16 mg/mL, 20 mg/mL, 25 mg/mL, or 30 mg/mL, which is not limited here.

Magnetic beads in the magnetic bead suspension are magnetic beads modified with silica hydroxyl or carboxyl groups. For example, a concentration of the magnetic bead suspension ranges from 10 mg/mL to 40 mg/mL. For example, the concentration of the magnetic bead suspension is 10 mg/mL, 15 mg/mL, 20 mg/mL, 25 mg/mL, 30 mg/mL, or 40 mg/mL, which is not limited here.

The first cleaning solution is mainly used to wash an adsorption carrier bound to nucleic acids to remove protein impurities. For example, the first cleaning solution includes: guanidine isothiocyanate, tris (hydroxymethyl)aminomethane hydrochloride, polyoxyethylene sorbitan monolaurate, sodium chloride, and isopropanol. Here, a molarity of guanidine isothiocyanate ranges from 0.5 mol/L to 3 mol/L, a molarity of tris (hydroxymethyl)aminomethane hydrochloride ranges from 0.5 mmol/L to 10 mmol/L, a weight by volume percent of polyoxyethylene sorbitan monolaurate ranges from 0.01% to 0.1%, a molarity of sodium chloride ranges from 0.5 mol/L to 2 mol/L, and a percent by volume of isopropanol ranges from 20% to 50%. pH of the first cleaning solution takes a value ranging from 6.5 to 8.0.

For example, the molarity of guanidine isothiocyanate is 0.5 mol/L, 1 mol/L, 1.5 mol/L, 2 mol/L, 2.5 mol/L, or 3 mol/L, which is not limited here.

For example, the molarity of tris (hydroxymethyl)aminomethane hydrochloride is 0.5 mmol/L, 1 mmol/L, 2 mmol/L, 3 mmol/L, 4 mmol/L, 5 mmol/L, 6 mmol/L, 7 mmol/L, 8 mmol/L, 9 mmol/L, or 10 mmol/L, which is not limited here.

Polyoxyethylene sorbitan monolaurate is used to increase the dispersion properties of the magnetic beads in an aqueous solution. For example, the weight by volume percent of polyoxyethylene sorbitan monolaurate is 0.01%, 0.02%, 0.03%, 0.04%, 0.05%, 0.06%, 0.07%, 0.08%, 0.09%, or 0.1%, which is not limited here.

Sodium chloride is used to maintain the ion concentration in the solution and provide ions that are adsorbed between the nucleic acids and the magnetic beads through ion bridges to prevent them from being washed away by the cleaning solution. For example, the molarity of sodium chloride is 0.5 mol/L, 1 mol/L, 1.5 mol/L, or 2 mol/L, which is not limited here.

Isopropanol is used to keep the nucleic acids in a dehydrated state in the solution to prevent them from being eluted by the cleaning solution. For example, the percent by volume of isopropanol is 20%, 30%, 40%, or 50%, which is not limited here.

For example, the pH of the first cleaning solution takes a value of 6.5, 6.6, 6.8, 7.0, 7.2, 7.3, 7.5, 7.8, or 8.0, which is not limited here.

The second cleaning solution includes sodium chloride and a second surfactant, where a molarity of sodium chloride ranges from 0.05 mol/L to 0.5 mol/L, and a weight by volume percent of the second surfactant ranges from 0.5% to 5%. pH of the second cleaning solution takes a value ranging from 6.9 to 7.1.

For example, the second surfactant includes at least one of: polyethylene glycol p-isooctylphenyl ether, polyoxyethylene sorbitan monolaurate, and ethylphenyl polyethylene glycol.

Ethylphenyl polyethylene glycol is abbreviated as NP-40, having the molecular formula of C₁₅H₂₄O·(C₂H₄O)ₙ, where n is a positive integer. Ethylphenyl polyethylene glycol is a commonly used nonionic non-denaturing detergent and surfactant.

The cleaning solution is used to wash the adsorption carrier bound to the nucleic acids to remove non-nucleic acid contaminants and salts, and to maintain a solution in which the nucleic acids are bound to the adsorption carrier.

For example, the molarity of sodium chloride is 0.05 mol/L, 0.1 mol/L, 0.15 mol/L, 0.25 mol/L, 0.3 mol/L, 0.45 mol/L, or 0.5 mol/L, which is not limited here.

For example, the weight by volume percent of the second surfactant is 0.5%, 0.8%, 1%, 1.5%, 2%, 2.5%, 3%, 4.5%, or 5%, which is not limited here.

The cleaning solution contains a sodium chloride solution with a high salt ion concentration and a solution with a low pH value, which can ensure that the nucleic acids will not be released from the magnetic beads into the solution; moreover, the second surfactant with a certain concentration can remove impurities on the surface of the magnetic beads, thereby achieving the purpose of purifying the nucleic acids.

Therefore, the above examples provide a non-alcohol cleaning solution capable of improving the long-term storage stability of the performance of kit consumables and reagents of the automatic extractor.

The elution solution is a solution for eluting the nucleic acids bound to the adsorption carrier. For example, the elution solution is a TE buffer solution or nuclease-free water.

The kit for nucleic acid extraction provided in the embodiments of the present disclosure includes the lysis/binding solution provided in the above embodiments. Therefore, the lysis process of the biological sample and the binding process of the nucleic acids and the magnetic beads can be unified, so that the sample can be bound to the magnetic beads while being lysed, thereby reducing the time of nucleic acid extraction and better matching the automatic nucleic acid extractor to realize fully automatic extraction.

In addition, the kit for nucleic acid extraction provided in the embodiments of the present disclosure further includes the red blood cell lysis solution, which uses the concentrated salt of the red blood cell lysis solution, and then the white blood cells are lysed after separation, which can prevent the nucleic acids from being contaminated in subsequent steps due to the introduction of a large amount of sugar and proteins on the surface of the red blood cells.

Furthermore, the kit for nucleic acid extraction provided in the embodiments of the present disclosure further includes the non-alcohol second cleaning solution, which can improve the long-term storage stability of the performance of kit consumables and reagents of the automatic extractor.

Based on the kit for nucleic acid extraction provided in the above embodiments, the embodiments of the present disclosure provide a method for nucleic acid extraction.

The method includes the following steps AC to E.

In step A0, a blood sample is put in a container and a red blood cell lysis solution is added in the container, the blood sample and the red blood cell lysis solution are mixed and centrifugation is performed thereon, and a supernatant is removed, so as to obtain a first precipitate.

For example, the container is a centrifuge tube.

For example, the blood sample is bovine blood.

For example, a volume of the red blood cell lysis solution is 3 to 5 times a volume of the blood sample. For example, the volume of the red blood cell lysis solution is 3, 3.5, 4, 4.5, or 5 times the volume of the blood sample, which is not limited here.

For example, the blood sample to which the red blood cell lysis solution has been added is mixed using a vortex mixer.

For example, in the centrifugation treatment step, a rotational speed of a centrifuge ranges from 10000 revolutions per minute (rpm) to 12000 rpm. For example, the rotational speed of centrifugation is 10000 rpm, 11000 rpm, or 12000 rpm, which is not limited here. A time of centrifugation ranges from 1 minute to 3 minutes. For example, the time of centrifugation is 1 minute, 2 minutes, or 3 minutes, which is not limited here.

By controlling the appropriate rotational speed and time of centrifugation, red blood cell debris is retained in the supernatant and removed.

For example, the steps of adding the red blood cell lysis solution, mixing the blood sample and the red blood cell lysis solution and performing centrifugation thereon, and removing the supernatant are repeated at least once This can more completely remove red blood cells, thereby preventing the nucleic acids from being contaminated in subsequent steps due to the introduction of a large amount of sugar and proteins on the surface of the red blood cells.

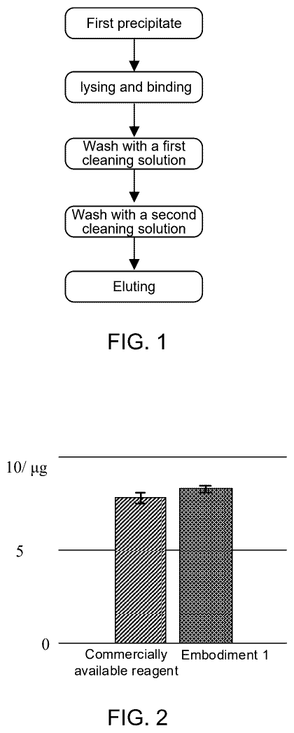

After the blood sample forms a first precipitate by lysing the red blood cells, as shown in FIG. 1, the method for nucleic acid extraction further includes the steps of: lysing and binding the first precipitate, washing with a first cleaning solution, washing with a second cleaning solution, and eluting. The steps of lysing and combining the first precipitate, washing with the first cleaning solution, washing with the second cleaning solution, and eluting can be matched with an automatic nucleic acid extractor to realize fully automatic extraction. The specific operations of each step are as follows.

The steps of lysis and binding include steps A and B.

In step A, a lysis/binding solution and a proteinase K solution are added to the first precipitate and the lysis/binding solution, the proteinase K solution and the sample are mixed, at a temperature condition of 55° C. to 80° C.

The first precipitate is also referred to as a sample to be performed with nucleic acid extraction.

This step is used to release nucleic acids from biological samples (such as viruses) in blood samples and destroy the protein structure of the biological samples (such as viruses), promote the separation of proteins and nucleic acids, and dehydrate nucleic acid molecules.

For example, the above temperature is 55° C., 60° C., 65° C., 70° C., 75° C., or 80° C., which is not limited here.

For example, a volume of the lysis/binding solution is 2 to 4 times a volume of the blood sample. For example, the volume of the lysis/binding solution is 2, 2.5, 3, 3.5, or 4 times the volume of the blood sample, which is not limited here.

For example, a volume of the proteinase K solution is 0.05 to 0.2 times the volume of the blood sample. For example, the volume of the proteinase K solution is 0.05 times, 0.1 times, 0.12 times, 0.15 times, or 0.2 times the volume of the blood sample, which is not limited here.

In step B, a magnetic bead suspension is added to a mixed solution obtained in step A, the magnetic bead suspension and the mixed solution are mixed, the magnetic bead suspension and the mixed solution are placed on a magnetic rack for standing, and a supernatant is removed, so as to obtain a second precipitate.

This step is used to effectively bind the nucleic acids to an adsorption carrier (such as magnetic beads).

For example, a volume of the magnetic bead suspension is 0.075 to 0.2 times the volume of the blood sample. For example, the volume of the magnetic bead suspension is 0.075 times, 0.095 times, 0.125 times, 0.15 times, or 0.2 times the volume of the blood sample, which is not limited here.

For example, the centrifuge tube is placed on the magnetic rack for standing for 2 minutes.

In step C, washing with the first cleaning solution, in which the first cleaning solution is added to the second precipitate, the first cleaning solution and the second precipitate are mixed, the first cleaning solution and the second precipitate are placed on a magnetic rack for standing, and a supernatant is removed, so as to obtain a third precipitate.

This step is used to wash the adsorption carrier bound to the nucleic acids to remove protein impurities.

For example, a volume of the first cleaning solution is 2.4 to 4.5 times the volume of the blood sample. For example, the volume of the first cleaning solution is 2.4 times, 2.7 times, 3.0 times, 3.3 times, 4 times, or 4.5 times the volume of the blood sample, which is not limited here.

For example, the centrifuge tube is placed on the magnetic rack for standing for 2 minutes.

In step D, washing with the second cleaning solution, in which the second cleaning solution is added to the third precipitate, the second cleaning solution and the third precipitate are mixed, the second cleaning solution and the third precipitate are placed on a magnetic rack for standing, and a supernatant is removed, so as to obtain a fourth precipitate.

This step is used to wash the adsorption carrier bound to the nucleic acids to remove non-nucleic acid contaminants and salts, and to maintain a solution in which the nucleic acids are bound to the adsorption carrier.

For example, a volume of the second cleaning solution is 2.4 to 4.5 times the volume of the blood sample. For example, the volume of the second cleaning solution is 2.4 times, 2.7 times, 3.0 times, 3.3 times, 4 times, or 4.5 times the volume of the blood sample, which is not limited here.

For example, the centrifuge tube is placed on the magnetic rack for standing for 2 minutes.

In step E, eluting, in which an eluent solution is added to the fourth precipitate and the eluent solution and the fourth precipitate are mixed, at a temperature condition of 40° C. to 60° C., the eluent solution and the fourth precipitate are placed on a magnetic rack for standing, and a supernatant is removed, so as to obtain extracted nucleic acids.

This step is used to elute the nucleic acids bound to the adsorption carrier.

For example, the above temperature is 40° C., 45° C., 48° C., 52° C., 55° C., or 60° C., which is not limited here.

For example, the centrifuge tube is placed on the magnetic rack for standing for 2 minutes.

The method for nucleic acid extraction provided by the embodiments of the present disclosure involves the lysis/binding solution provided by the above embodiments. Therefore, in the process of nucleic acid extraction, after adding the blood sample, the lysis process of the biological sample and the binding process of the nucleic acids and the magnetic beads can be unified, so that the sample can be bound to the magnetic beads while being lysed, thereby reducing the time of nucleic acid extraction and better matching the automatic nucleic acid extractor to realize fully automatic extraction.

Moreover, before matching the automatic nucleic acid extractor to realize the nucleic acid extraction, the above method further includes the step of red blood cell lysis, that is, the red blood cells are first swelled and lysed using the concentrated salt of the red blood cell lysis solution, and then the white blood cells are lysed after separation, which can prevent the nucleic acids from being contaminated in subsequent steps due to the introduction of a large amount of sugar and proteins on the surface of the red blood cells.

Based on the kit for nucleic acid extraction and the method for nucleic acid extraction provided in the above embodiments, the following specific embodiments are provided.

### Embodiment 1

The performance of nucleic acids extracted by the kit for nucleic acid extraction provided in the embodiments of the present disclosure is compared with the performance of nucleic acids extracted by a commercially available kit for nucleic acid extraction, where the performance of nucleic acids includes the concentration and purity of nucleic acids.

The reagent components of the kit for nucleic acid extraction provided in the embodiments of the present disclosure are as follows.

(1) Red blood cell lysis solution: 5 mmol/L sodium chloride (NaCl), 5% polyoxyethylene sorbitan monolaurate (Tween 20) at weight by volume percent, 320 mmol/L glucose, and 10 mmol/L tris (hydroxymethyl)aminomethane (Tris), where the pH of tris (hydroxymethyl)aminomethane (Tris) takes a value of 8.2.

(2) Lysis/binding solution: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium phosphate at weight by volume percent.

That is, in Embodiment 1, the protein denaturant is guanidine isothiocyanate, the non-alcohol dehydrating agent is sodium dodecyl sulfate (SDS), the first surfactant is polyoxyethylene sorbitan monolaurate (Tween 20), the buffer solution is a TE buffer solution with the pH value of 7.2, and the chaotropic salt is ammonium phosphate.

(3) Proteinase K solution: with a concentration of 20 mg/mL.

(4) First cleaning solution: 1 mol/L guanidine isothiocyanate, 1 mmol/L tris (hydroxymethyl)aminomethane hydrochloride (Tris-HCl), 0.05% polyoxyethylene sorbitan monolaurate (Tween 20) at weight by volume percent, 1 mol/L sodium chloride (NaCl), and 40% isopropanol at a percent by volume. The pH of the first cleaning solution takes a value of 7.5.

(5) Second cleaning solution: 0.5 mol/L sodium chloride (NaCl), and 0.5% polyethylene glycol-p-isooctylphenyl ether (Triton 100) at weight by volume percent. The pH of the second cleaning solution takes a value of 7.0.

(6) Eluent solution: a TE buffer solution.

In this embodiment, bovine whole blood is used as a blood sample, and the specific operation process of nucleic acid extraction is as follows, including steps of removing red blood cells from the blood sample and extracting nucleic acids.

The steps of removing red blood cells from the blood sample include: steps S1 to S4.

In step S1, 250 microliters (μL) of the blood sample is taken in a 2 mL centrifuge tube, and 800 μL of the red blood cell lysis solution is added and mixed with sufficient shaking for 10 seconds to obtain a clear red transparent mixture.

A volume of the above cell lysis solution is 3.2 times a volume of the blood sample.

In step S2, centrifugation is performed on the above mixture at 12000 revolutions per minute (rpm) for 1 minute, and a supernatant is removed to obtain a white precipitate.

In step S3, 800 μL of the red blood cell lysis solution is added to the centrifuge tube in step S2, and a mixed solution is obtained by resuspending the precipitate with sufficient shaking.

In step S4, centrifugation is performed on the mixed solution obtained in step S3 at 12000 revolutions per minute (rpm) for 1 minute, and a supernatant is removed to obtain a first precipitate.

The steps of extracting nucleic acids further include: steps S5 to S10.

In step S5, 600 μL of the lysis/binding solution is added to the first precipitate at a temperature condition of 65° C., mixed using a vortex mixer, and left to stand for 10 minutes.

In step S6, 20 μL of the magnetic bead suspension is added to a mixed solution obtained in step S5, mixed using a vortex mixer, left to stand for 10 minutes, and then mixed using the vortex mixer for 3 seconds at 2-minute intervals.

A volume of the magnetic bead suspension is 0.08 times the volume of the blood sample.

In step S7, a mixed solution obtained in step S6 is placed on a magnetic rack for standing for 2 minutes, and a supernatant is removed to obtain a second precipitate.

Before placing the mixed solution obtained in step S6 on the magnetic rack, the mixed solution obtained in step S6 may be subjected to low-speed centrifugation to accelerate the subsequent deposition of magnetic beads.

In step S8, 600 μL of the first cleaning solution is added to the second precipitate and mixed manually; then, the centrifuge tube is placed on a magnetic rack for standing-adsorption for 2 minutes, and a supernatant is removed to obtain a third precipitate.

A volume of the first cleaning solution is 2.4 times the volume of the blood sample.

For example, manual mixing includes: flicking the outside of the centrifuge tube with a finger or inverting the centrifuge tube back and forth up and down.

In step S9, 600 μL of the second cleaning solution is added to the third precipitate and mixed manually; then, the centrifuge tube is placed on a magnetic rack and let to stand for adsorption for 2 minutes, a supernatant is removed to obtain a fourth precipitate, and the precipitate is dried at room temperature until there is no obvious liquid on the surface of the magnetic beads.

A volume of the second cleaning solution is 2.4 times the volume of the blood sample.

In step S10, 100 μL of the elution solution is added in the centrifuge tube and shaken for eluting at a temperature condition of 56° C. for 10 minutes; then, the centrifuge tube is placed on a magnetic rack for standing for 2 minutes, and a supernatant is transferred to a new centrifuge tube to obtain extracted nucleic acids.

The concentration of the nucleic acids extracted in the above example is tested by Qubit 4.0 Nucleic Acid/Protein Fluorescence Quantifier, and the purity of the nucleic acids is tested by a micro-spectrophotometer (nanodrop).

It will be noted that when using the micro-spectrophotometer (nanodrop), A260/A280 is used to evaluate the purity of nucleic acids, and the larger the value, the higher the purity of the nucleic acids.

The test results of the concentration and purity of the nucleic acids are compared with the performance of a commercially available reagent.

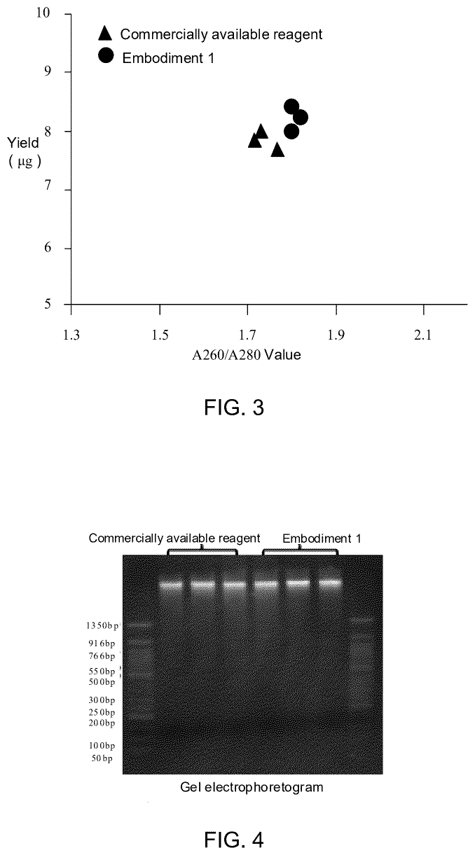

Table 1 is a comparison of the performance of nucleic acids extracted in Embodiment 1 and nucleic acids extracted by a commercially available kit. FIG. 2 is a histogram showing the efficiency of nucleic acid extraction. FIG. 3 is a scattergram showing the purity of nucleic acid extraction. As can be seen from the data results, the reagent system provided by the present scheme is slightly better than the commercially available reagents as a comparator in terms of DNA extraction yield and purity. FIG. 4 is a gel electropherogram for nucleic acid extraction, where the ordinate represents the gel electrophoresis migration rate (bp is an abbreviation of base pair). It can be seen from FIG. 4 that Embodiment 1 and the commercially available reagent are not much different in terms of the DNA band distribution. It will be noted that the DNA band distribution of the nucleic acid sample extracted by the commercially available kit for nucleic acid extraction indicates that the nucleic acids are not degraded during the experiment. The nucleic acid sample extracted in Embodiment 1 is not much different from the nucleic acid sample extracted by the commercially available kit for nucleic acid extraction in terms of the DNA band distribution, indicating that the nucleic acid sample extracted by the kit for nucleic acid extraction provided in Embodiment 1 of the present disclosure does not degrade during the experiment and can be used for downstream experiments.

Embodiment 1 shows that the kit for nucleic acid extraction provided by the embodiments of the present disclosure can allow the sample to be lysed and bound to magnetic beads at the same time, and the performance of extracted nucleic acids is better than that of the nucleic acids extracted by the commercially available reagent.

TABLE 1
Comparison of the performance of nucleic acids extracted in Embodiment
1 and nucleic acids extracted by a commercially available kit

| | DNA Recovery | | Extraction |
|---|---|---|---|
| | Concentration | DNA Purity | Time |
| Kit | (ng/μL) | (A260/280) | (minutes) |
| Commercially | 106/112/111 | 1.70/1.71/1.72 | 50 |
| available reagent | | | |
| Embodiment 1 | 114/118/117 | 1.80/1.80/1.82 | 50 |

It will be noted that as shown in Table 1, the three numbers in each table cell represent data obtained by repeating the experiment three times under the same experimental conditions. For example, the numbers “106/112/111” of DNA recovery concentration (ng/μL) of the commercially available reagent mean that the nucleic acids are extracted three times using the commercially available reagent, and the concentration of the nucleic acids is tested each time using Qubit 4.0 Nucleic Acid/Protein Fluorescence Quantifier, and in the three experimental results, the DNA recovery concentrations are 106 ng/μL, 112 ng/μL and 111 ng/μL, respectively. The same applies to the following.

### Embodiment 2

The reagent components of this embodiment are similar to those of Embodiment 1. The reagent components are pre-filled into reagent slot holes in the automatic nucleic acid extractor. The reagents corresponding to the slot holes are shown in Table 2 below.

Among them, the proteinase K solution needs to be refrigerated and stored at a temperature condition of 2° C. to 8° C., and added to hole 2 separately before the reaction to ensure that the proteinase K has sufficient activity.

TABLE 2
Correspondence between reagent components and reagent
slot holes in the automatic nucleic acid extractor

| | | | | Magnetic | | | |
|---|---|---|---|---|---|---|---|
| | | | Mixing | Attraction | | Temperature | Liquid |
| | | | Time | Time | Mix | Reaction | Volume |
| Step | Hole | Step | (minutes) | (seconds) | Speed | (° C.) | (μL) |
| 1 | 2 | Lysis | 10 | 0 | Middle | 65 | 900 |
| 2 | 1 | Magnetic | 0 | 15 | Middle | Closed | 100 |
| | | bead transfer | | | | | |
| 3 | 2 | Nucleic acid | 10 | 15 | Middle | Closed | 900 |
| | | capture | | | | | |
| 4 | 3 | First | 1 | 15 | Middle | Closed | 700 |
| | | cleaning | | | | | |
| | | solution | | | | | |
| 5 | 4 | Second | 1 | 15 | Middle | Closed | 600 |
| | | cleaning | | | | | |
| | | solution | | | | | |
| 6 | 5 | Nucleic acid | 5 | 30 | Middle | 56 | 100 |
| | | elution | | | | | |
| 7 | 1 | Magnetic | 1 | 0 | Middle | Closed | 100 |
| | | bead release | | | | | |

It will be noted that the lysis in step 1 here corresponds to the lysis in step S5 in Embodiment 1. Before step 1, Embodiment 2 further includes a step of manually removing red blood cells from the blood sample, and the specific operation steps are the same as steps S1 to S4 of Embodiment 1.

After obtaining the first precipitate by adopting steps S1 to S4, the first precipitate needs to be transferred to the reagent slot hole 2 of the automatic nucleic acid extractor. The specific operation steps include: mixing the first precipitate with 200 μL of phosphate buffered saline (PBS) buffer solution, and transferring the mixed solution to the reagent slot hole 2 of the automatic nucleic acid extractor. After that, the machine is turned on for running, the parameters are set as described in Table 1, and the automatic extraction is initiated at the end of the setup. At the end of the run of the automatic nucleic acid extractor, 80 μL of the nucleic acid eluate solution in reagent slot hole 5 is taken in a new centrifuge tube and used for the next test.

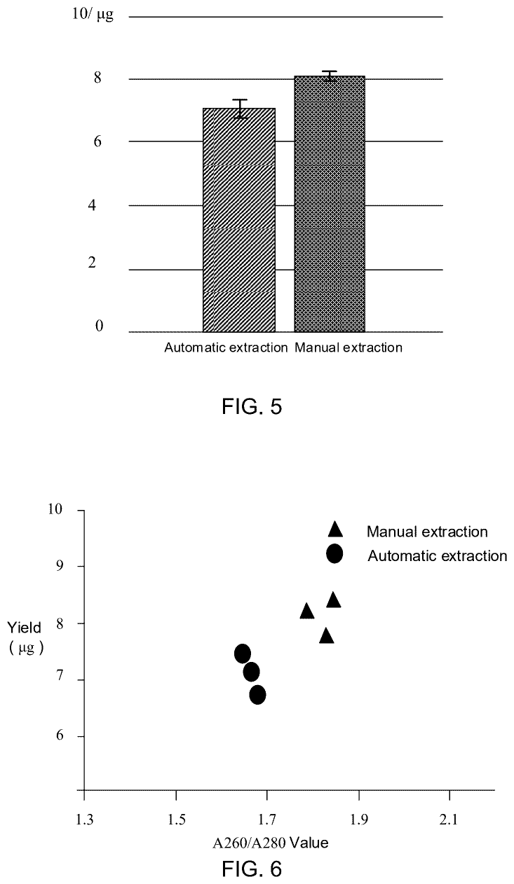

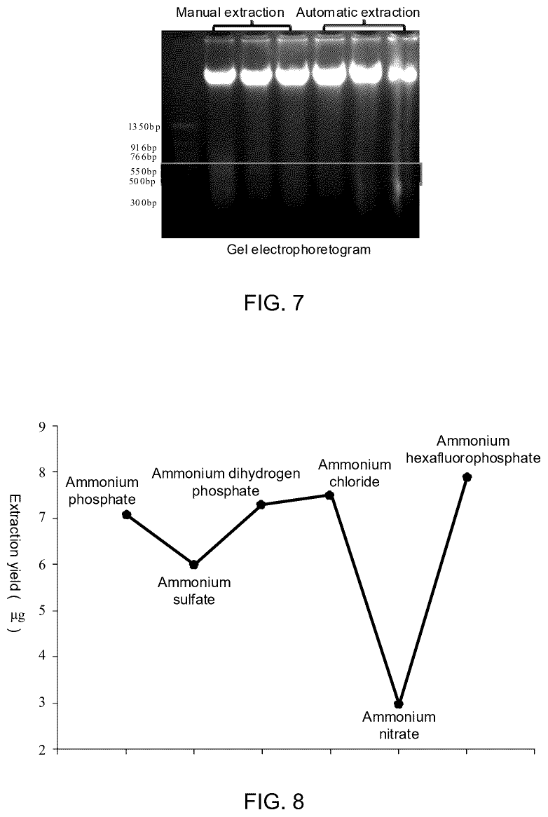

The performance of the nucleic acid sample manually extracted in Embodiment 1 is compared with that of the nucleic acid sample automatically extracted in Embodiment 2. Table 3 is a comparison of the effects of a manual extraction method and an automatic extraction method on nucleic acid performance. FIG. 5 is a histogram showing the efficiency of nucleic acid extraction. FIG. 6 is a scattergram showing the purity of nucleic acid extraction. It can be seen from Table 3, FIG. 5 and FIG. 6 that the automatic extraction method is not as good as manual extraction in yield and purity of extraction. FIG. 7 is a gel electropherogram for nucleic acid extraction. It can be seen from the figure that there is not much difference in DNA band distribution, indicating that the nucleic acid sample extracted manually and the nucleic acid sample extracted automatically do not degrade during the experiment.

TABLE 3
Comparison of the effects of a manual extraction method and
an automatic extraction method on nucleic acid performance

| | DNA Recovery | | Extraction |
|---|---|---|---|
| | Concentration | DNA Purity | Time |
| Extraction Method | (ng/μL) | (A260/280) | (minutes) |
| Manual extraction | 117/111/120 | 1.79/1.83/1.85 | 35 |
| Automatic extraction | 96/106/101.5 | 1.68/1.65/1.67 | 30 |

It can be seen from Embodiment 2 that the lysis/binding solution for nucleic acid extraction and the kit containing the lysis/binding solution provided in the embodiments of the present disclosure can be matched with an automatic nucleic acid extractor to realize fully automatic extraction. Although the automatic extraction method is not as good as the manual extraction method in terms of yield and purity of extraction, it does not affect the performance of extracted nucleic acids.

### Embodiment 3

This embodiment is used to verify the effects of different types of anions in the chaotropic salt on the concentration and purity of extracted nucleic acids.

After the chaotropic salt dissociates into an ionic salt solution in an aqueous solution, hydrogen bonds in the water will gather around the salt ions, driving the water molecules to gather. This will cause a disturbance in the arrangement of the hydrogen bonds in the aqueous solution, thereby strengthening the internal interactions of ionic organic matters (such as DNA) and causing coagulation. Different ionic salts have different interferences on water-soluble hydrogen bonds. According to the Hofmeister sequence, different anions and different cations are selected for comparison in this embodiment. By affecting other components in the lysis solution, such as proteinase K and protein, a group of ion pairs is selected that have the optimal DNA precipitation effect and do not cause strong interference with protein digestion.

It will be noted that the Hofmeister sequence refers to the change in an aggregation state of a polymer, which can be achieved by simply adding specific ions, where different ions have different abilities to precipitate polymers.

Embodiment 3 uses the automatic nucleic acid extractor to extract nucleic acids, where the operation process can be referred to Embodiment 2.

To verify the effects of different types of anions in the chaotropic salt on the concentration and purity of extracted nucleic acids, the following examples are provided.

Example 3.1: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium phosphate at weight by volume percent. The pH of the lysis/binding solution takes a value of 7.2.

Example 3.2: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium sulfate at weight by volume percent. The pH of the lysis/binding solution takes a value of 7.2.

Example 3.3: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium dihydrogen phosphate at weight by volume percent. The pH of the lysis/binding solution takes a value of 7.2.

Example 3.4: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium chloride at weight by volume percent. The pH of the lysis/binding solution takes a value of 7.2.

Example 3.5: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium nitrate at weight by volume percent. The pH of the lysis/binding solution takes a value of 7.2.

Example 3.6: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium hexafluorophosphate at weight by volume percent. The pH of the lysis/binding solution takes a value of 7.2.

That is to say, for Example 3.1 to Example 3.6, the chaotropic salt components of the lysis/binding solution added to the reagent slot hole 2 are different, specifically, the cations of the chaotropic salts are all ammonium ions (NH₄⁺), and the anions of the chaotropic salts are different, and the specific chaotropic salt components used are ammonium phosphate, ammonium sulfate, ammonium dihydrogen phosphate, ammonium chloride, ammonium nitrate, and ammonium hexafluorophosphate.

TABLE 4
Comparison of the effects of different anions in the chaotropic
salt on the performance of nucleic acid extraction

| | DNA Recovery | |
|---|---|---|
| | Concentration | DNA Purity |
| Chaotropic Salt Component | (ng/μL) | (A260/280) |
| Ammonium phosphate | 96/106/101.5 | 1.68/1.65/1.67 |
| Ammonium sulfate | 84/79/94 | 1.66/1.63/1.67 |
| Ammonium dihydrogen phosphate | 101/93/89 | 1.70/1.72/1.69 |
| Ammonium chloride | 110/104/107 | 1.68/1.71/1.64 |
| Ammonium nitrate | 38/43/47 | 1.58/1.52/1.57 |
| Ammonium hexafluorophosphate | 113/115/109 | 1.73/1.75/1.74 |

Table 4 is a comparison of the effects of different anions in the chaotropic salt on the performance of nucleic acid extraction. FIG. 8 is a line graph of the yield of nucleic acid extraction. From the test results, it can be seen that among the six different anionic chaotropic salt components, the concentration and purity of nucleic acids extracted by the lysis/binding solution containing ammonium hexafluorophosphate are optimal.

### Embodiment 4

This embodiment is used to verify the effects of different types of cations in the chaotropic salt on the concentration and purity of extracted nucleic acids.

Embodiment 4 uses the automatic nucleic acid extractor to extract nucleic acids, where the operation process can be referred to Embodiment 2.

To verify the effects of different types of cations in the chaotropic salt on the concentration and purity of extracted nucleic acids, the following examples are provided.

Example 4.1: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% calcium hexafluorophosphate at weight by volume percent. The pH of the lysis/binding solution takes a value of 7.2.

Example 4.2: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% magnesium hexafluorophosphate at weight by volume percent. The pH of the lysis/binding solution takes a value of 7.2.

Example 4.3: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% potassium hexafluorophosphate at weight by volume percent. The pH of the lysis/binding solution takes a value of 7.2.

Example 4.4: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% sodium hexafluorophosphate at weight by volume percent. The pH of the lysis/binding solution takes a value of 7.2.

Example 4.5: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% ammonium hexafluorophosphate at weight by volume percent. The pH of the lysis/binding solution takes a value of 7.2.

Example 4.6: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% N-ethylpyridinium hexafluorophosphate at weight by volume percent. The pH of the lysis/binding solution takes a value of 7.2.

That is to say, for Example 4.1 to Example 4.6, the chaotropic salt components of the lysis/binding solution added to the reagent slot hole 2 are different, specifically, the anions of the chaotropic salts are all hexafluorophosphate ions (PF₆⁻), and the cations of the chaotropic salts are different, and the specific chaotropic salt components used are calcium hexafluorophosphate, magnesium hexafluorophosphate, potassium hexafluorophosphate, sodium hexafluorophosphate, ammonium hexafluorophosphate, and N-ethylpyridinium hexafluorophosphate.

TABLE 5
Comparison of the effects of different cations in the chaotropic
salt on the performance of nucleic acid extraction

| | DNA Recovery | |
|---|---|---|
| | Concentration | DNA Purity |
| Chaotropic Salt Component | (ng/μL) | (A260/280) |
| Calcium hexafluorophosphate | 45/53/46 | 1.53/1.49/1.51 |
| Magnesium hexafluorophosphate | 36/39/44 | 1.46/1.53/1.57 |
| Potassium hexafluorophosphate | 76/79/82 | 1.64/1.71/1.69 |
| Sodium hexafluorophosphate | 96/102/103 | 1.76/1.73/1.68 |
| Ammonium hexafluorophosphate | 113/115/109 | 1.73/1.75/1.74 |
| N-ethylpyridinium | 117/119/116 | 1.79/1.82/1.83 |
| hexafluorophosphate | | |

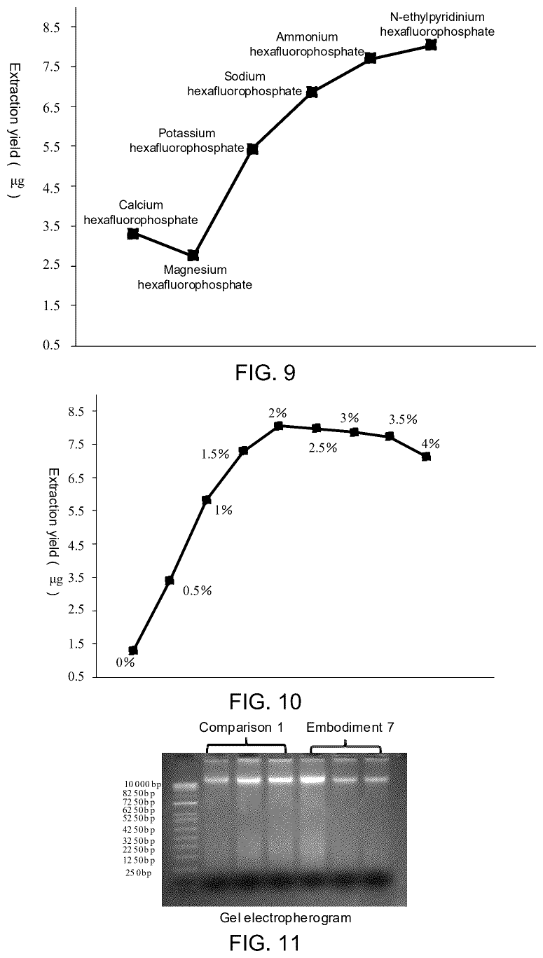

Table 5 is a comparison of the effects of different cations in the chaotropic salt on the performance of nucleic acid extraction. FIG. 9 is a line graph of the yield of nucleic acid extraction. From the test results, it can be seen that different cations have different effects on the extraction performance, in which the concentration and purity of nucleic acids extracted by the lysis/binding solution containing N-ethylpyridinium cationic chaotropic salt are optimal.

### Embodiment 5

This embodiment is used to verify the effects of the concentration of the chaotropic salt on the concentration and purity of extracted nucleic acids.

This embodiment uses the automatic nucleic acid extractor to extract nucleic acids, where the operation process can be referred to Embodiment 2.

It can be seen from Embodiment 3 and Embodiment 4 that the concentration and purity of the nucleic acids extracted by the lysis/binding solution containing the chaotropic salt component of N-ethylpyridinium hexafluorophosphate are relatively good. Therefore, in the lysis/binding solution, N-ethylpyridinium hexafluorophosphate is used as the component of the chaotropic salt.

In order to verify the effects of the concentration of the chaotropic salt on the concentration and purity of extracted nucleic acids, the following examples are provided.

Example 5.1: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), and 0.5% sodium dodecyl sulfate (SDS) at weight by volume percent. The pH of the lysis/binding solution takes a value of 7.2.

Example 5.2: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 0.5% N-ethylpyridinium hexafluorophosphate at weight by volume percent. The pH of the lysis/binding solution takes a value of 7.2.

Example 5.3: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 1% N-ethylpyridinium hexafluorophosphate at weight by volume percent. The pH of the lysis/binding solution takes a value of 7.2.

Example 5.4: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 1.5% N-ethylpyridinium hexafluorophosphate at weight by volume percent. The pH of the lysis/binding solution takes a value of 7.2.

Example 5.5: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 7.2.

Example 5.6: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2.5% N-ethylpyridinium hexafluorophosphate at weight by volume percent. The pH of the lysis/binding solution takes a value of 7.2.

Example 5.7: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3% N-ethylpyridinium hexafluorophosphate at weight by volume percent. The pH of the lysis/binding solution takes a value of 7.2.

Example 5.8: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 3.5% N-ethylpyridinium hexafluorophosphate at weight by volume percent. The pH of the lysis/binding solution takes a value of 7.2.

Example 5.9: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 4% N-ethylpyridinium hexafluorophosphate at weight by volume percent. The pH of the lysis/binding solution takes a value of 7.2.

That is to say, for Example 5.1 to Example 5.9, the weight by volume percents of the chaotropic salt (N-ethylpyridinium hexafluorophosphate) of the lysis/binding solution added to reagent slot hole 2 are different, and the specific weight by volume percents of the chaotropic salt used are 0%, 0.5%, 1%, 1.5%, 2%, 2.5%, 3%, 3.5%, and 4%, respectively.

TABLE 6
Comparison of the effects of different concentrations of the
chaotropic salt on the performance of nucleic acid extraction

| | DNA Recovery | |
|---|---|---|
| Chaotropic Salt | Concentration | DNA Purity |
| Concentration | (ng/μL) | (A260/280) |
| 0% | 15/23/18 | 1.53/1.49/1.51 |
| 0.5% | 44/49/53 | 1.46/1.53/1.57 |
| 1% | 76/85/88 | 1.64/1.71/1.69 |
| 1.5% | 107/100/105 | 1.76/1.73/1.68 |
| 2% | 118/111/116 | 1.73/1.75/1.74 |
| 2.5% | 110/114/117 | 1.79/1.82/1.83 |
| 3% | 113/115/109 | 1.73/1.75/1.74 |
| 3.5% | 108/109/114 | 1.72/1.68/1.70 |
| 4% | 101/105/99 | 1.77/1.74/1.76 |

Table 6 is a comparison of the effects of different concentrations of the chaotropic salt on the performance of nucleic acid extraction. FIG. 10 is a line graph of the yield of nucleic acid extraction. It can be seen from Table 6 and FIG. 10 that in a case where the weight by volume percent of the chaotropic salt in the lysis/binding solution is 2%, the performance of extracted nucleic acids is relatively good.

### Embodiment 6

This embodiment is used to verify the effects of different pH values of the lysis/binding solution on the concentration and purity of extracted nucleic acids.

This embodiment uses the automatic nucleic acid extractor to extract nucleic acids, where the operation process can be referred to Embodiment 2.

In order to verify the effects of different pH values of the lysis/binding solution on the concentration and purity of extracted nucleic acids, the following examples are provided.

Example 6.1: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 4.4.

Example 6.2: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 5.4.

Example 6.3: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 6.4.

Example 6.4: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 7.4.

Example 6.5: the components of the lysis/binding solution include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 8.4.

That is to say, for Example 6.1 to Example 6.5, the pH values of the lysis/binding solution added to the reagent slot hole 2 are different, and the pH values of the lysis/binding solution are 4.4, 5.4, 6.4, 7.4, and 8.4, respectively.

TABLE 7
Comparison of the effects of different pH values of the lysis/binding
solution on the performance of nucleic acid extraction

| | DNA Recovery | |
|---|---|---|
| | Concentration | DNA Purity |
| pH Value | (ng/μL) | (A260/280) |
| 4.4 | 32/36/31 | 1.48/1.51/1.53 |
| 5.4 | 44/48/43 | 1.61/1.63/1.67 |
| 6.4 | 67/73/66 | 1.62/1.67/1.69 |
| 7.4 | 116/109/115 | 1.76/1.81/1.83 |
| 8.4 | 38/41/46 | 1.71/1.74/1.72 |
| 4.4 | 32/36/31 | 1.48/1.51/1.53 |

Table 7 is a comparison of the effects of different pH values of the lysis/binding solution on the performance of nucleic acid extraction. It can be seen from the table that in a case where the pH of the lysis/binding solution takes a value of 7.4, the concentration and purity of extracted nucleic acids are relatively good. It will be noted that when verifying the effects of different pH values of the lysis/binding solution on the performance of nucleic acid extraction, a difference between the pH values of the lysis/binding solution is designed to be 1. Due to deviations in reagent preparation, test results of the nucleic acid performance in a case where the pH of the lysis/binding solution takes a value of 7.2 are not much different from those in a case where the pH of the lysis/binding solution takes a value of 7.4. That is to say, in a case where the pH of the lysis/binding solution takes a value of about 7.4, the concentration and purity of extracted nucleic acids are also relatively good.

Based on the data of Embodiment 3 regarding the effects of different types of anions in the chaotropic salt on the concentration and purity of extracted nucleic acids, Embodiment 4 regarding the effects of different types of cations in the chaotropic salt on the concentration and purity of extracted nucleic acids, Embodiment 5 regarding the effects of the concentration of the chaotropic salt on the concentration and purity of extracted nucleic acids, and Embodiment 6 regarding the effects of different pH values of the lysis/binding solution on the concentration and purity of extracted nucleic acids, the following examples of relatively good lysis/binding solutions are provided.

For example, the components of the lysis/binding solution include: 5 mol/L guanidine isothiocyanate, 0.5% sodium dodecyl sulfate at weight by volume percent, 0.1% polyoxyethylene sorbitan monolaurate at weight by volume percent, 100 mmol/L TE buffer solution, 10 mmol/L ethylenediaminetetraacetic acid, and 2% N-ethylpyridinium hexafluorophosphate at weight by volume percent. The pH of the lysis/binding solution takes a value of 7.4.

For example, the components of the lysis/binding solution include: 5 mol/L guanidine isothiocyanate, 0.5% sodium dodecyl sulfate at weight by volume percent, 0.1% polyoxyethylene sorbitan monolaurate at weight by volume percent, 100 mmol/L TE buffer solution, 10 mmol/L ethylenediaminetetraacetic acid, and 2% N-ethylpyridinium hexafluorophosphate at weight by volume percent. The pH of the lysis/binding solution takes a value of 7.2.

### Embodiment 7

This embodiment is used to verify the effects of using a lysis/binding solution without isopropanol and a binding solution with isopropanol on the concentration and purity of extracted nucleic acids.

The reagent components of the lysis/binding solution in this embodiment are: 5 mol/L guanidine isothiocyanate, 0.1% polyoxyethylene sorbitan monolaurate (Tween 20) at weight by volume percent, 100 mmol/L TE buffer solution, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent. The pH of the lysis/binding solution takes a value of 7.2.

The other components of the kit are the same as those in Embodiment 1, and the nucleic acid extraction steps are the same as those in Embodiment 1.

The comparison for Embodiment 7 (referred to as Comparison 1) is a reagent with isopropanol in the binding solution.

The reagent components of the kit of Comparison 1 are as follows.

(1) Red blood cell lysis solution: 5 mmol/L sodium chloride (NaCl), 5% polyoxyethylene sorbitan monolaurate (Tween 20) at weight by volume percent, 320 mmol/L glucose, and 10 mmol/L tris (hydroxymethyl)aminomethane (Tris) with the pH value of 8.2.

(2) Lysis solution: 3.5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.5, 10 mmol/L ethylenediaminetetraacetic acid (EDTA), 0.5% sodium dodecyl sulfate (SDS) at weight by volume percent, and 1% polyethylene glycol-p-isooctylphenyl ether (Triton 100) at weight by volume percent.

(3) Proteinase K solution, includes: a proteinase K solution with a concentration of 10 mg/mL, and further includes 50 mmol/L tris (hydroxymethyl)aminomethane (Tris) with pH value of 8.0, 5 mmol/L calcium chloride (CaCl₂)), and 20% glycerol at a percent by volume.

(4) Binding solution: 0.5 mmol/L sodium chloride (NaCl), 45% isopropanol at a percent by volume, 15% PEG800 at weight by volume percent, 100 mmol/L tris (hydroxymethyl)aminomethane (Tris), where the pH of tris (hydroxymethyl)aminomethane (Tris) takes a value of 8.0, and 5 mmol/L ethylenediaminetetraacetic acid (EDTA).

Here, PEG 800 (Polyethylene Glycol 800) is polyethylene glycol with an average molecular weight of 800.

(5) First cleaning solution: 1 mol/L guanidine isothiocyanate, 1 mmol/L tris (hydroxymethyl)aminomethane hydrochloride (Tris-HCl), 0.05% polyoxyethylene sorbitan monolaurate (Tween 20) at weight by volume percent, 1 mol/L sodium chloride (NaCl), and 40% isopropanol at a percent by volume. The pH of the first cleaning solution takes a value of 7.5.

(6) Second cleaning solution: 0.5 mol/L sodium chloride (NaCl), and 0.5% polyethylene glycol-p-isooctylphenyl ether (Triton 100) at weight by volume percent. The pH of the second cleaning solution takes a value of 7.0.

(7) Eluent solution: a TE buffer solution.

In Comparison 1, bovine whole blood is used as a blood sample, and the specific operation process of nucleic acid extraction is as follows, including steps of removing red blood cells from the blood sample and extracting nucleic acids.

The steps of removing red blood cells from the blood sample are the same as those in Embodiment 1, which specifically include: steps R1 to R4.

In step R1, 250 μL of the blood sample is taken in a 2 mL centrifuge tube, and 800 μL of the red blood cell lysis solution is added and mixed with sufficient shaking for 10 seconds to obtain a clear red transparent mixture.

A volume of the above cell lysis solution is 3.2 times a volume of the blood sample.

In step R2, centrifugation is performed on the above mixture at 12000 revolutions per minute (rpm) for 1 minute, and a supernatant is removed to obtain a white precipitate.

In step R3, 800 μL of the red blood cell lysis solution is added to the centrifuge tube in step R2, and a mixed solution is obtained by resuspending the precipitate with sufficient shaking.

In step R4, centrifugation is performed on the mixed solution obtained in step R3 at 12000 revolutions per minute (rpm) for 1 minute, and a supernatant is removed to obtain a first precipitate.

The steps of extracting nucleic acids further include: steps R5 to R12.

In step R5, 400 μL of the lysis solution is added to the first precipitate at a temperature condition of 65° C., mixed using a vortex mixer, and left to stand for 30 minutes.

In step R6, 400 μL of isopropanol is added to a mixed solution in step R5, mixed using a vortex mixer.

In step R7, 20 μL of the magnetic bead suspension is added to a mixed solution obtained in step R6, mixed using a vortex mixer, left to stand for 5 minutes, and then mixed using the vortex mixer for 3 seconds at 2-minute intervals.

In step R8, 600 μL of the binding solution is added to a mixed solution obtained in step R7, mixed using a vortex mixer, left to stand for 10 minutes, and then mixed using the vortex mixer for 3 seconds at 2-minute intervals.

In step R9, a mixed solution obtained in step R8 is placed on a magnetic rack for standing for 2 minutes, and a supernatant is removed to obtain a second precipitate.

Before placing the mixed solution obtained in step R8 on the magnetic rack, the mixed solution obtained in step R8 may be subjected to low-speed centrifugation to accelerate the deposition of magnetic beads in subsequent steps.

In step R10, 600 μL of the first cleaning solution is added to the second precipitate and mixed manually; then, the centrifuge tube is placed on a magnetic rack for standing-adsorption for 2 minutes, and a supernatant is removed to obtain a third precipitate.

In step R11, 600 μL of the second cleaning solution is added to the third precipitate and mixed manually; then, the centrifuge tube is placed on a magnetic rack and let to stand for adsorption for 2 minutes, a supernatant is removed to obtain a fourth precipitate, and the precipitate is dried at room temperature until there is no obvious liquid on the surface of the magnetic beads.

In step R12, 100 μL of the elution solution is added in the centrifuge tube and shaken for eluting at a temperature condition of 56° C. for 10 minutes; then, the centrifuge tube is placed on a magnetic rack for standing for 2 minutes, and a supernatant is transferred to a new centrifuge tube to obtain extracted nucleic acids.

TABLE 8
Comparison of the effects of the extraction methods
using a lysis/binding solution without isopropanol
and using a binding solution with isopropanol on
the performance of extracted nucleic acids

| | DNA Recovery | |
|---|---|---|
| | Concentration | DNA Purity |
| Extraction Reagent | (ng/μL) | (A260/280) |
| Lysis/binding solution | 108/113/117 | 1.77/1.78/1.80 |
| without isopropanol | | |
| Binding solution with | 118/112/111 | 1.75/1.72/1.74 |
| isopropanol | | |

Table 8 is a comparison of the effects of the extraction methods using the lysis/binding solution without isopropanol and using the binding solution with isopropanol on the performance of extracted nucleic acids. It can be seen from the test results that in comparison of the performance of nucleic acids obtained using the extraction method using the lysis/binding solution without isopropanol of Embodiment 7 and the extraction method using the binding solution with isopropanol of Comparison 1, there is not much difference in the concentration, and there is an increase in the purity of the nucleic acids obtained by the extraction of the lysis/binding solution without isopropanol. FIG. 11 is a gel electropherogram for nucleic acid extraction. From the figure, it can be seen that there is not much difference in the distribution of the DNA bands, indicating that none of the nucleic acid samples obtained by the extraction method using the lysis/binding solution without isopropanol of Embodiment 7 and the extraction method using the binding solution with isopropanol of Comparison 1 are degraded during the experimental process.

In order to verify the effects of the second cleaning solution containing ethanol on the performance of extracted nucleic acids, the following Comparison 2 is provided.

Comparison 2 is compared with Embodiment 7.

The reagent components of the kit of Comparison 2 are as follows.

(1) Red blood cell lysis solution: 5 mmol/L sodium chloride (NaCl), 5% polyoxyethylene sorbitan monolaurate (Tween 20) at weight by volume percent, 320 mmol/L glucose, and 10 mmol/L tris (hydroxymethyl)aminomethane (Tris) with the pH value of 8.2.

(2) Lysis/binding solution: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), 0.5% sodium dodecyl sulfate (SDS) at weight by volume percent, and 2% N-ethylpyridinium hexafluorophosphate at weight by volume percent.

(3) Proteinase K solution, includes: a proteinase K solution with a concentration of 10 mg/mL, and further includes 50 mmol/L tris (hydroxymethyl)aminomethane (Tris) with pH value of 8.0, 5 mmol/L calcium chloride (CaCl₂)), and 20% glycerol at a percent by volume.

(4) First cleaning solution: 1 mol/L guanidine isothiocyanate, 1 mmol/L tris (hydroxymethyl)aminomethane hydrochloride (Tris-HCl), 0.05% polyoxyethylene sorbitan monolaurate (Tween 20) at weight by volume percent, 1 mol/L sodium chloride (NaCl), and 40% isopropanol at a percent by volume. The pH of the first cleaning solution takes a value of 7.5.

(5) Second cleaning solution: a TE buffer solution and an 80% ethanol solution at a percent by volume.

(6) Eluent solution: a TE buffer solution.

TABLE 9
Comparison of the effects of the extraction methods
using a second cleaning solution without isopropanol
and using a second cleaning solution with isopropanol
on the performance of extracted nucleic acids

| | DNA Recovery | |
|---|---|---|
| | Concentration | DNA Purity |
| Extraction Reagent | (ng/μL) | (A260/280) |
| Second cleaning solution | 106/109/113 | 1.78/1.80/1.83 |
| without isopropanol | | |
| Second cleaning solution | 114/107/112 | 1.71/1.68/1.76 |
| with isopropanol | | |

Table 9 is a comparison of the effects of the extraction methods using a second cleaning solution without isopropanol and using a second cleaning solution with isopropanol on the performance of extracted nucleic acids. From the test results, it can be seen that the extraction methods using the second cleaning solution without isopropanol in Embodiment 7 and using the second cleaning solution with isopropanol in Comparison 2 have little effect on the performance of extracted nucleic acids.

Therefore, under the condition of ensuring the performance of extracted nucleic acids, using the second cleaning solution without isopropanol can improve the performance of long-term storage of the reagent.

In order to compare the effects of a lysis/binding solution with a chaotropic salt and a lysis/binding solution without a chaotropic salt on the performance of extracted nucleic acids, the following Comparison 3 is provided.

Comparison 3 is compared with Embodiment 7.

The components of the lysis/binding solution in Comparison 3 include: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), and 0.5% sodium dodecyl sulfate (SDS) at weight by volume percent.

The other components of the kit are the same as those in Embodiment 1 (it can be understood that the other components of the kit are also the same as those in Embodiment 7), and the nucleic acid extraction steps are the same as those in Embodiment 1.

TABLE 10
Comparison of the effects of the extraction methods using a lysis/binding
solution without chaotropic salt and a lysis/binding solution with
chaotropic salt on the performance of extracted nucleic acids

| | DNA Recovery | |
|---|---|---|
| | Concentration | DNA Purity |
| Extraction Reagent | (ng/μL) | (A260/280) |
| Lysis/binding solution | 108/103/111 | 1.72/1.70/1.70 |
| without chaotropic salt | | |
| Lysis/binding solution | 121/117/116 | 1.79/1.86/1.83 |
| with chaotropic salt | | |

Table 10 is a comparison of the effects of the extraction methods using a lysis/binding solution without chaotropic salt and a lysis/binding solution with chaotropic salt on the performance of extracted nucleic acids. From the test results, the concentration and purity of the nucleic acids extracted by the extraction method using the lysis/binding solution with chaotropic salt are higher than the concentration and purity of the nucleic acids extracted by the extraction method using the lysis/binding solution without chaotropic salt in Comparison 3.

Therefore, Comparative Example 3 proves that the chaotropic salt is helpful for the extraction of nucleic acid.

### Embodiment 8

This embodiment is to verify the effects of different types of chaotropic salts on the concentration and purity of extracted nucleic acids, and to compare the performance of nucleic acids extracted by a commercially available reagent.

The reagent components of the lysis/binding solution provided in this embodiment are: 5 mol/L guanidine isothiocyanate, 0.1% polyoxyethylene sorbitan monolaurate (Tween 20) at weight by volume percent, 100 mmol/L TE buffer solution, where the pH of the TE buffer solution takes a value of 7.2, 10 mmol/L ethylenediaminetetraacetic acid (EDTA), and 0.5% sodium dodecyl sulfate (SDS) at weight by volume percent, and 2% chaotropic salt at weight by volume percent.

The difference is that the chaotropic salt components in the lysis/binding solution are different, and the chaotropic salt components are: sodium hexafluorophosphate, ammonium hexafluorophosphate, 1-allyl-3-methylimidazolium hexafluorophosphate, N-ethylpyridinium hexafluorophosphate, 1-Allyl-3-methylimidazolium chloride, N-ethylpyridinium chloride, 1-allyl-3-methylimidazolium phosphate, and N-ethylpyridinium phosphate.

The other components of the kit are the same as those in Embodiment 1, and the nucleic acid extraction steps are the same as those in Embodiment 1.

TABLE 11
Effects of different types of chaotropic salts on the performance
of extracted nucleic acids and comparison with the performance of
nucleic acids extracted using a commercially available reagent

| | DNA Recovery | |
|---|---|---|
| | Concentration | DNA Purity |
| Extraction Reagent | (ng/μL) | (A260/280) |
| Commercially available reagent | 108/110/114 | 1.75/1.76/1.72 |
| Sodium hexafluorophosphate | 103/109/110 | 1.73/1.75/1.75 |
| Ammonium hexafluorophosphate | 114/121/107 | 1.76/1.81/1.73 |
| 1-allyl-3-methylimidazolium | 106/103/109 | 1.74/1.78/1.77 |
| hexafluorophosphate | | |
| N-ethylpyridinium | 116/123/127 | 1.77/1.82/1.83 |
| hexafluorophosphate | | |
| 1-Allyl-3-methylimidazolium | 88/79/81 | 1.73/1.65/1.66 |
| chloride | | |
| N-ethylpyridinium chloride | 111/112/115 | 1.76/1.74/1.76 |
| 1-allyl-3-methylimidazolium | 68/56/59 | 1.73/1.71/1.68 |
| phosphate | | |
| N-ethylpyridinium phosphate | 74/79/82 | 1.72/1.65/1.70 |

Table 11 shows the effects of different types of chaotropic salts on the performance of extracted nucleic acids, and compares the performance of nucleic acids extracted with the commercially available reagent. From the test results, in a case where the chaotropic salt in the lysis/binding solution is sodium hexafluorophosphate, ammonium hexafluorophosphate, 1-allyl-3-methylimidazolium hexafluorophosphate, N-ethylpyridinium hexafluorophosphate, or N-ethylpyridinium chloride, the performance of extracted nucleic acids is not much different from that of the nucleic acids extracted with the commercially available reagent.

Therefore, unifying the components of the lysis solution and the binding solution and selecting an appropriate chaotropic salt not only reduce the time of nucleic acid extraction, but also allow the sample to bind to the magnetic beads while being lysed, and can better match the automatic nucleic acid extractor to realize fully automatic extraction without affecting the performance of extracted nucleic acids.

The foregoing description is only specific embodiments of the present disclosure, but the scope of protection of the present disclosure is not limited thereto. Any changes or replacements that a person skilled in the art could conceive of within the technical scope of the present disclosure shall be included in the protection scope of the present disclosure. Therefore, the protection scope of the present disclosure shall be subject to the protection scope of the claims.

1. A lysis/binding solution for nucleic acid extraction, the lysis/binding solution comprising:
a protein denaturant, a non-alcohol dehydrating agent, a first surfactant, a buffer solution, ethylenediaminetetraacetic acid, and a chaotropic salt, wherein
a molarity of the protein denaturant ranges from 3.5 mol/L to 5.5 mol/L, a weight by volume percent of the non-alcohol dehydrating agent ranges from 0.1% to 1%, a weight by volume percent of the first surfactant ranges from 0.05% to 0.5%, a molarity of the buffer solution ranges from 5 mmol/L to 150 mmol/L, a molarity of the ethylenediaminetetraacetic acid ranges from 5 mmol/L to 20 mmol/L, and a weight by volume percent of the chaotropic salt ranges from 1% to 8%.

2. The lysis/binding solution for nucleic acid extraction according to claim 1, wherein anions in the chaotropic salt comprise at least one type of: PO₄³⁻, SO₄²⁻, H₂PO₄⁻, HCOO⁻, Cl⁻, NO₃⁻, CF₃COO⁻, BF₄⁻, ClO₄⁻, and PF₆⁻.

3. The lysis/binding solution for nucleic acid extraction according to claim 1, wherein cations in the chaotropic salt comprise at least one type of: Ca²⁺, Mg²⁺, Li⁺, Na⁺, K⁺, NH₄⁺, an imidazolium cation, and an N-ethylpyridinium cation.

4. The lysis/binding solution for nucleic acid extraction according to claim 1, wherein the chaotropic salt comprises at least one of: ammonium phosphate, ammonium sulfate, ammonium dihydrogen phosphate, ammonium chloride, calcium hexafluorophosphate, potassium hexafluorophosphate, sodium hexafluorophosphate, ammonium hexafluorophosphate, 1-allyl-3-methylimidazolium hexafluorophosphate, N-ethylpyridinium hexafluorophosphate, and N-ethylpyridinium chloride.

5. The lysis/binding solution for nucleic acid extraction according to claim 1, wherein the non-alcohol dehydrating agent comprises at least one of:
polyoxyethylene lauryl ether, polyvinyl carbazole, polyvinyl alcohol, polyacrylamide, polyacrylic acid, polyoxyethylene sorbitan monolaurate, and sodium dodecyl sulfate.

6. The lysis/binding solution for nucleic acid extraction according to claim 1, wherein the protein denaturant comprises at least one of: guanidine isothiocyanate and guanidine hydrochloride.

7. The lysis/binding solution for nucleic acid extraction according to claim 1, wherein the buffer solution comprises at least one of: a Tris-EDTA (TE) buffer solution and tris (hydroxymethyl)aminomethane, the TE buffer solution comprising tris (hydroxymethyl)aminomethane hydrochloride and ethylenediaminetetraacetic acid.

8. The lysis/binding solution for nucleic acid extraction according to claim 1, wherein the first surfactant comprises polyoxyethylene sorbitan monolaurate or polyethylene glycol-p-isooctylphenyl ether.

9. The lysis/binding solution for nucleic acid extraction according to claim 1, wherein pH of the lysis/binding solution takes a value ranging from 4.4 to 7.4.

10. The lysis/binding solution for nucleic acid extraction according to claim 1, wherein the protein denaturant is guanidine isothiocyanate, the non-alcohol dehydrating agent is sodium dodecyl sulfate, the first surfactant is polyoxyethylene sorbitan monolaurate, the buffer solution is a TE butter solution, and the chaotropic salt is N-ethylpyridinium hexafluorophosphate, wherein
a molarity of the guanidine isothiocyanate is 5 mol/L, a weight by volume percent of the sodium dodecyl sulfate is 0.5%, a weight by volume percent of the polyoxyethylene sorbitan monolaurate is 0.1%, a molarity of the TE buffer solution is 100 mmol/L, the molarity of the ethylenediaminetetraacetic acid is 10 mmol/L, and a weight by volume percent of the N-ethylpyridinium hexafluorophosphate is 2%; and pH of the lysis/binding solution takes a value of 7.4.

11. The lysis/binding solution for nucleic acid extraction according to claim 1, wherein pH of the buffer solution takes a value ranging from 7.0 to 7.4.

12. (canceled)

13. A cleaning solution for nucleic acid extraction, the cleaning solution comprising: sodium chloride and a second surfactant, wherein a molarity of the sodium chloride ranges from 0.05 mol/L to 0.5 mol/L, and a weight by volume percent of the second surfactant ranges from 0.5% to 5%; and pH of the cleaning solution takes a value ranging from 6.9 to 7.1.

14. The cleaning solution for nucleic acid extraction according to claim 13, wherein the second surfactant comprises at least one of: polyethylene glycol p-isooctylphenyl ether, polyoxyethylene sorbitan monolaurate, and ethylphenyl polyethylene glycol.

15. A kit for nucleic acid extraction, the kit comprising: the lysis/binding solution for nucleic acid extraction according to claim 1; and
a second cleaning solution, comprising sodium chloride and a second surfactant, wherein a molarity of the sodium chloride ranges from 0.05 mol/L to 0.5 mol/L, and a weight by volume percent of the second surfactant ranges from 0.5% to 5%; and pH of the second cleaning solution takes a value ranging from 6.9 to 7.1.

16. The kit for nucleic acid extraction according to claim 15, further comprising: a red blood cell lysis solution, wherein
the red blood cell lysis solution comprises: sodium chloride, polyoxyethylene sorbitan monolaurate, glucose, and tris (hydroxymethyl)aminomethane, wherein a molarity of the sodium chloride ranges from 1 mol/L to 10 mol/L, a weight by volume percent of the polyoxyethylene sorbitan monolaurate ranges from 3% to 6%, a molarity of the glucose ranges from 200 mmol/L to 500 mmol/L, and a molarity of the tris (hydroxymethyl)aminomethane ranges from 1 mmol/L to 50 mmol/L; and
pH of the red blood cell lysis solution takes a value ranging from 8.0 to 8.4.

17. The kit for nucleic acid extraction according to claim 15, further comprising: a proteinase K solution with a concentration ranging from 10 mg/mL to 30 mg/mL; and/or
further comprising: a magnetic bead suspension with a concentration ranging from 10 mg/mL to 40 mg/mL.

18. (canceled)

19. The kit for nucleic acid extraction according to claim 15, further comprising:
a first cleaning solution, comprising: guanidine isothiocyanate, tris (hydroxymethyl)aminomethane hydrochloride, polyoxyethylene sorbitan monolaurate, sodium chloride, and isopropanol, wherein a molarity of the guanidine isothiocyanate ranges from 0.5 mol/L to 3 mol/L, a molarity of the tris (hydroxymethyl)aminomethane hydrochloride ranges from 0.5 mmol/L to 10 mmol/L, a weight by volume percent of the polyoxyethylene sorbitan monolaurate ranges from 0.01% to 0.1%, a molarity of the sodium chloride ranges from 0.5 mol/L to 2 mol/L, and a percent by volume of the isopropanol ranges from 20% to 50%; and pH of the first cleaning solution takes a value ranging from 6.5 to 8.0; and/or
an eluent solution, wherein the eluent solution comprises: a TE buffer solution.

20. (canceled)

21. A method for nucleic acid extraction, the method comprising:
adding a lysis/binding solution and a proteinase K solution to a sample to be performed with nucleic acid extraction and mixing the lysis/binding solution, the proteinase K solution and the sample to form a mixed solution, at a temperature condition of 55° C. to 80° C.;
adding a magnetic bead suspension to the mixed solution, mixing the magnetic bead suspension and the mixed solution, placing the magnetic bead suspension and the mixed solution on a magnetic rack for standing, and removing a supernatant to obtain a second precipitate;
adding a first cleaning solution to the second precipitate, mixing the first cleaning solution and the second precipitate, placing the first cleaning solution and the second precipitate on a magnetic rack for standing, and removing a supernatant, to obtain a third precipitate;
adding a second cleaning solution to the third precipitate, mixing the second cleaning solution and the third precipitate, placing the second cleaning solution and the third precipitate on a magnetic rack for standing, and removing a supernatant to obtain a fourth precipitate; and
adding an eluent solution to the fourth precipitate and mixing the eluent solution and the fourth precipitate, at a temperature condition of 40° C. to 60° C., placing the eluent solution and the fourth precipitate on a magnetic rack for standing, and removing a supernatant to obtain extracted nucleic acids.

22. The method for nucleic acid extraction according to claim 21, wherein a volume of the lysis/binding solution is 2 to 4 times a volume of a blood sample, a volume of the proteinase K solution is 0.05 to 0.2 times the volume of the blood sample, a volume of the magnetic bead suspension is 0.075 to 0.2 times the volume of the blood sample, a volume of the first cleaning solution is 2.4 to 4.5 times the volume of the blood sample, and a volume of the second cleaning solution is 2.4 to 4.5 times the volume of the blood sample; and the sample to be performed with nucleic acid extraction is obtained through processing the blood sample.

23. The method for nucleic acid extraction according to claim 21, further comprising: adding the lysis/binding solution and the proteinase K solution to the sample and mixing the lysis/binding solution, the proteinase K solution and the sample, putting a blood sample in a container and adding a red blood cell lysis solution in the container, mixing the blood sample and the red blood cell lysis solution and performing centrifugation thereon, and removing a supernatant to obtain a first precipitate, the sample to be performed with nucleic acid extraction being the first precipitate;
wherein a rotational speed of centrifugation ranges from 10000 rpm to 12000 rpm, and a time of centrifugation ranges from 1 minute to 3 minutes; and adding the red blood cell lysis solution, mixing the blood sample and the red blood cell lysis solution and performing centrifugation thereon, and removing the supernatant are repeated at least once; and
a volume of the red blood cell lysis solution is 3 to 5 times a volume of the blood sample.
